(19) United States
(12) Patent Application Publication
(10) Pub. No.: US 2011/0294841 A1
(43) Pub. Date: Dec. 1, 2011

(54) NOVEL STATIN PHARMACEUTICAL COMPOSITIONS AND RELATED METHODS OF TREATMENT

(76) Inventors: Hector GUZMAN, Jamaica Plain, MA (US); Örn ALMARSSON, Shrewsbury, MA (US); Julius REMENAR, Framington, MA (US)

(21) Appl. No.: 13/161,154

(22) Filed: Jun. 15, 2011

Related U.S. Application Data
(63) Continuation of application No. 12/184,888, filed on Aug. 1, 2008, now abandoned.
Application No. 12/184,888 is a continuation of application No. PCT/US2007/061360, filed on Jan. 31, 2007.
Application No. 12/184,888 is a continuation-in-part of application No. 11/197,880, filed on Aug. 5, 2005, now Pat. No. 7,642,287.
(60) Provisional application No. 60/764,075, filed on Feb. 1, 2006.
(60) Provisional application No. 60/599,543, filed on Aug. 6, 2004.
(60) Provisional application No. 60/623,518, filed on Oct. 29, 2004.
(60) Provisional application No. 60/655,982, filed on Feb. 24, 2005.

(51) Int. Cl. A61K 31/505; A61K 31/40; A61P 7/00; A61K 31/4418; A61K 31/225; A61K 31/405; A61K 31/351

(52) U.S. Cl. 514/275; 514/419; 514/423; 514/460; 514/277; 514/510

(57) ABSTRACT

The invention provides novel omega-3 oil formulations of one or more statins. These formulations are readily bioavailable. Notably, because the formulations of the invention contain an omega-3 oils as the major ingredient, they not only provide an antihypercholesterolemic effect due to the statin active ingredient, they also provide recommended daily dosages of omega-3 oils (i.e., approximately 1 gram of omega-3 oil per day), or a portion thereof. The invention also provides novel salts of one or more statins.

### RELATED APPLICATIONS

This application is a continuation application of pending U.S. application Ser. No. 12/184,888, filed Aug. 1, 2008, which is a continuation application of International Appln. No. PCT/US2007/061360, filed Jan. 31, 2007, which claims priority to U.S. Provisional Appln. No. 60/764,075, filed Feb. 1, 2006. U.S. application Ser. No. 12/184,888 is also a continuation-in-part application of U.S. application Ser. No. 11/197,880, filed Aug. 5, 2005, which claims priority to U.S. Provisional Appln. Nos. 60/559,543, filed Aug. 6, 2004, 60/623,518, filed Oct. 29, 2004, and 60/655,982, filed Feb. 24, 2005. The contents of each of these applications are incorporated by reference herein, in their entirety and for all purposes.

### FIELD OF THE INVENTION

The invention provides novel omega-3 oil suspensions of statins. The invention also provides novel statin salts and pharmaceutical formulations comprising the same.

### BACKGROUND OF THE INVENTION

It has been clear for several decades that elevated blood cholesterol is a major risk factor for coronary heart disease (CHD), and many studies have shown that the risk of CHD events can be reduced by lipid-lowering therapy. Prior to 1987, the lipid-lowering armamentarium was limited essentially to a low saturated fat and cholesterol diet, the bile acid sequestrants (cholestyramine and colestipol), nicotinic acid (niacin), the fibrates and probucol. Unfortunately, all of these treatments have limited efficacy or tolerability, or both. With the introduction of lovastatin (MEVACOR®; see U.S. Pat. No. 4,231,938), the first inhibitor of HMG-CoA reductase to become available for prescription in 1987, physicians were able to obtain comparatively large reductions in plasma cholesterol with very few adverse effects.

In addition to the natural fermentation products, mevastatin and lovastatin, there are now a variety of semi-synthetic and totally synthetic HMG-CoA reductase inhibitors, including simvastatin (ZOCOR®); see U.S. Pat. No. 4,444,784), pravastatin sodium salt (PRAVACHOL®; see U.S. Pat. No. 4,346,227), fluvastatin sodium salt (LESCOL®; see U.S. Pat. No. 5,354,772), atorvastatin calcium salt (LIPITOR®; see U.S. Pat. No. 5,273,995) and cerivastatin sodium salt (also known as rivastatin; see U.S. Pat. No. 5,177,080). The HMG-CoA reductase inhibitors described above belong to a structural class of compounds which contain a moiety which can exist as either a 3-hydroxy lactone ring or as the corresponding ring opened dihydroxy open-acid, and are often referred to as “statins.”

Salts of the dihydroxy open-acid can be prepared, and in fact, as noted above, several of the marketed statins are administered as the dihydroxy open acid salt forms. Lovastatin and simvastatin are marketed worldwide in their lactonized form.

The hypotriglyceridemic effects of omega-3 oils from fish oils are well established. Amounts both above and below about 1 gram per day of omega-3 oils from fish oil have been shown to decrease serum triglyceride concentrations by about 25% to about 40%, decrease VLDL blood plasma levels, and to increase both LDL and HDL plasma levels (See e.g., Harris, William S, Clin. Cardiol. 22, (Suppl. II), 11-40-11-43 (1999)). A dose-response relationship exists between omega-3 oil intake and triglyceride lowering. Postprandial triglyceridemia is especially sensitive to chronic omega-3 oil consumption. Kris-Etherton, et al., Circulation, 2002;106:2747.

While there are numerous known statin dosage forms, the need continues to exist for commercially practicable statin pharmaceutical compositions and formulations that exhibit enhanced bioavailability, arc readily formulated and administered, and comprise ingredients that enhance the antihypercholesterolemic effect of the statin.

### SUMMARY OF THE INVENTION

The invention provides novel omega-3 oil pharmaceutical formulations of one or more statins having unexpected properties. These pharmaceutical formulations are readily bioavailable. Notably, because the formulations of the invention contain an omega-3 oil as the major ingredient, they not only provide an antihypercholesterolemic effect due to the statin active ingredient, they also provide recommended daily dosages of omega-3 oils (i.e., one gram of omega-3 oil per day, as per AHA guidelines), or a portion thereof.

The invention comprises a suspension, or a heterogeneous formulation, of one or more statins in omega-3 oil. In specific embodiments, the invention provides suspensions of amorphous and/or crystalline particles of one or more statins in an omega-3 oil.

In one embodiment, pharmaceutical formulations of the invention comprise an omega-3 oil, wherein the omega-3 oil is an omega-3 alkyl ester, such as an omega-3 ethyl ester. In another embodiment, pharmaceutical formulations of the invention comprise an omega-3 mono-, di-, or triglyceride oil.

In another embodiment, the invention provides a pharmaceutical formulation comprising about 500, 600, 700, 800, 900, 1000, 1100, 1200, 1300, 1400, or 1500 mg of an omega-3 oil with greater than or equal to about 90 percent purity and about 1, 2, 3, 4, 5, 10, 15, 20, 25, 30, 35, 40, 45, 50, 55, 60, 65, 70, 75, 80, 90, 100, 110, 120, 130, 140, 150, or 160 mg of one or more salts of a statin(s). In another embodiment, the invention provides a pharmaceutical formulation comprising about 500, 600, 700, 800, 900, 1000, 1100, 1200, 1300, 1400, or 1500 mg of an omega-3 oil with a composition greater than or equal to about 90 percent EPA and DHA and about 1, 2, 3, 4, 5, 10, 15, 20, 25, 30, 35, 40, 45, 50, 55, 60, 65, 70, 75,80, 90, 100, 110, 120, 130, 140, 150, or 160 mg of one or more salts of a statin(s).

In another embodiment, the salt is a calcium salt of pravastatin. In another embodiment, the salt is a calcium salt of fluvastatin. In another embodiment, the salt is a magnesium salt of pravastatin. In another embodiment, the salt is a zinc salt of pravastatin. In another embodiment, the salt is crystalline.

In another embodiment, the invention provides a pharmaceutical formulation comprising about 500, 600, 700, 800, 900, 1000, 1100, 1200, 1300, 1400, or 1500 mg of an omega-3 oil with greater than or equal to about 90 percent purity and about 1, 2, 3, 4, 5, 10, 15, 20, 25, 30, 35, 40, 45, 50, 55, 60, 65, 70, 75, 80, 90, 100, 110, 120, 130, 140, 150, or 160 mg of one or more statins. In another embodiment, the invention provides a pharmaceutical formulation comprising about 500, 600, 700, 800, 900, 1000, 1100, 1200, 1300, 1400, or 1500 mg of an omega-3 oil with a composition greater than or equal to about 90 percent EPA and DHA and about 1, 2, 3, 4, 5, 10, 15, 20, 25, 30, 35, 40, 45, 50, 55, 60, 65, 70, 75, 80, 90, 100, 110, 120, 130, 140, 150, or 160 mg of one or more statins.

In another embodiment, the omega-3 oil is an omega-3 ester. In another embodiment, the omega-3 oil is an omega-3 ethyl ester. In another embodiment, the statin is in the form of a lactone. In another embodiment, the statin is in the form of a free acid.

In another embodiment, the present invention provides a salt of a statin. In another embodiment, the present invention provides a salt of pravastatin or fluvastatin. In a specific embodiment, a calcium salt of pravastatin is provided. In another specific embodiment, a magnesium salt of pravastatin is provided. In another specific embodiment, a zinc salt of pravastatin is provided. In another specific embodiment, a calcium salt of fluvastatin is provided. In another embodiment, a divalent salt of a statin is provided. In a specific embodiment, a divalent salt of pravastatin or fluvastatin is provided. In another embodiment, the salt of a statin is amorphous. In another embodiment, the salt of a statin is crystalline.

In another embodiment, a pharmaceutical formulation or a medicament comprising a salt of a statin is provided. In another embodiment, a pharmaceutical formulation or a medicament comprising a salt of a statin and an omega-3 oil is provided.

In another embodiment, the present invention provides a method for preparing a salt of a statin.

In another embodiment, a method for preparing a salt of a statin comprises:

(a) combining a statin and a salt in solution;

(b) initiating precipitation of a salt of said statin; and

(c) collecting said salt of said statin.

In another embodiment, the statin in step (a) can be a salt. For example, the statin in step (a) can be an alkali metal salt of a statin, such as, but not limited to, pravastatin sodium salt or fluvastatin sodium salt. In another embodiment, the salt in step (a) can be an alkaline earth metal salt. For example, the salt in step (a) can be a calcium or a magnesium salt, such as, but not limited to, calcium acetate or calcium chloride.

In another embodiment, a method of preventing, reducing, and/or treating hypercholesterolemia, atherosclerosis, hyperlipidemia, mixed dyslipidemia, cardiovascular events and disease including coronary events and cerebrovascular events, and coronary artery disease and/or cerebrovascular disease is provided by administering a pharmaceutical formulation of the present invention to a mammal in need of such prevention, reduction, and/or treatment.

These and other embodiments are described in greater detail in the following detailed description.

### BRIEF DESCRIPTION OF THE FIGURES

FIG. 1 shows a PXRD diffractogram of a pravastatin calcium salt.

FIG. 2 shows a TGA thermogram of a pravastatin calcium salt.

FIG. 3 shows an IR spectrum of a pravastatin calcium salt

FIG. 4 shows a DVS moisture sorption isotherm plot of a pravastatin calcium salt.

FIG. 5 shows a DVS moisture sorption isotherm plot of a pravastatin sodium salt.

FIG. 6 shows a PXRD diffractogram of a fluvastatin calcium salt.

FIG. 7 shows a DSC thermogram of a fluvastatin calcium salt.

FIG. 8 shows a TGA thermogram of a fluvastatin calcium salt.

FIG. 9 shows a Raman spectrum of a fluvastatin calcium salt.

FIG. 10 shows an IR spectrum of a fluvastatin calcium salt.

FIG. 11 shows a PXRD diffractogram of a pravastatin magnesium salt (habit A).

FIG. 12 shows a DSC thermogram of a pravastatin magnesium salt (habit A).

FIG. 13 shows a TGA thermogram of a pravastatin magnesium salt (habit A).

FIG. 14 shows an IR spectrum of a pravastatin magnesium salt (habit A).

FIG. 15 shows a DVS moisture sorption isotherm plot of a pravastatin magnesium salt (habit A).

FIG. 16 shows a PXRD diffractogram of a pravastatin magnesium salt (habit B).

FIG. 17 shows a DSC thermogram of a pravastatin magnesium salt (habit B).

FIG. 18 shows a TGA thermogram of a pravastatin magnesium salt (habit B).

FIG. 19 shows an IR spectrum of a pravastatin magnesium salt (habit B).

FIG. 20 shows a PXRD diffractogram of a pravastatin magnesium salt.

FIG. 21 shows a DSC thermogram of a pravastatin magnesium salt.

FIG. 22 shows a TGA thermogram of a pravastatin magnesium salt.

FIG. 23 shows a PXRD diffractogram of a pravastatin zinc salt.

FIG. 24 shows a DSC thermogram of a pravastatin zinc salt.

FIG. 25 shows a TGA thermogram of a pravastatin zinc salt.

FIG. 26 shows an IR spectrum of a pravastatin zinc salt.

FIG. 27 shows a Raman spectrum of a pravastatin zinc salt.

FIG. 28 shows a DVS moisture sorption isotherm plot of a pravastatin zinc salt.

FIG. 29 shows the stability data (percent lactone) of several pravastatin salts at 4 degrees C.

FIG. 30 shows the stability data (percent lactone) of several pravastatin salts at 40 degrees C.

FIG. 31 shows the stability data (percent other degradants) of several pravastatin salts at 40 degrees C.

### DETAILED DESCRIPTION OF THE INVENTION

The invention provides novel omega-3 oil pharmaceutical formulations of one or more statins having unexpected properties. These pharmaceutical formulations are readily bioavailable. Notably, because the formulations of the invention contain an omega-3 oil as the major ingredient, they not only provide an antihypercholesterolemic effect due to the statin active ingredient, they also provide recommended daily dosages of omega-3 oils (i.e., one gram of omega-3 oil per day, as per AHA guidelines), or a portion thereof.

The invention comprises a suspension, or a heterogeneous formulation, of one or more statins in omega-3 oil. In specific embodiments, the invention provides suspensions of amorphous and/or crystalline particles of one or more statins in an omega-3 oil.

In another embodiment, the present invention provides a salt of a statin. In another embodiment, the present invention provides a salt of pravastatin or fluvastatin. In a specific embodiment, a calcium salt of pravastatin is provided. In another specific embodiment, a magnesium salt of pravastatin is provided. In another specific embodiment, a zinc salt of pravastatin is provided. In another specific embodiment, a calcium salt of fluvastatin is provided. In another embodiment, a divalent salt of a statin is provided. In a specific embodiment, a divalent salt of pravastatin or fluvastatin is provided. In another embodiment, the salt of a statin is amorphous. In another embodiment, the salt of a statin is crystalline.

An “omega-3 oil” is any oil comprising omega-3 fatty acids, omega-3 mono-, di-, or triglycerides, or omega-3 esters, including, but not limited to, omega-3 alkyl esters. Omega-3 oils can be characterized using two unique descriptors, species and component. The species of an omega-3 oil is determined by the structure of the polyunsaturated carbon chain bound to the carboxyl group. The component of an omega-3 oil is determined by the chemical nature of the carboxyl group. For example, omega-3 fatty acids employ a —COOH structure bound to the polyunsaturated carbon chain, omega-3 esters employ a —COOR structure bound to the polyunsaturated carbon chain, and omega-3 mono- di- or tri-glycerides employ a —COOR′ structure bound to the polyunsaturated carbon chain, where R′ comprises a glycerol backbone. Oil composition can be described as both the species and the component(s) of an oil. For example, E681010 comprises about 68% EPA and about 10% DHA (mass percent) as ethyl esters. The remaining portion consists essentially of omega-3 oils other than EPA and DHA and other non-omega-3 oils. Such omega-3 oils can be found in, for example, fish oil, marine mammal fat, cod liver oil, walnuts and walnut oil, wheat germ oil, rapeseed oil, soybean lecithin derived oils, soybean derived oils, tofu derived oils, common bean derived oils, butternut derived oils, seaweed derived oils, flax-borage oil, and flax seed oil. Several omega-3 oils which can be used in making formulations of the invention include, but are not limited to, omega-3 oils such as Omegabrite® (Omega Natural Science), Epanova™ (Tillotts Pharma AG), OMEGA-3/90 (K D Pharma), Epax® (Pronova Biocare AS), and Incromega (Croda/Bioriginal).

“EPA” is defined as eicosapentaenoic acid (C20:5), and “DHA” is defined as docosahexaenoic acid (C22:6). Both EPA and DHA denote only the species of omega-3 oil and do not describe whether the components of such oils exist as, for example, triglycerides, diglycerides, monoglycerides, free acids, esters, or salts.

Specific omega-3 alkyl esters include the ethyl esters of EPA and DHA. For example, the E681010, OMEGA-3/90 (K D Pharma), and Incromega (Croda/Bioriginal) omega-3 ethyl esters are potential omega-3 alkyl esters.

Pharmaceutical formulations and medicaments may be described as mixtures of two or more components “by volume,” which is herein defined as the volume due to one component divided by the volume of all components of the, formulation. This ratio may be converted to or reported as a percentage of the total formulation volume. Such a quantity may also be indicated by “v/v” or “percent v/v.” Similarly, the phrases “by weight” and “by mass” describe the weight or mass due to one component divided by the weight or mass of all components of the formulation. This ratio may be converted to or reported as a percentage of the total formulation weight or mass. Such a quantity may also be indicated by “w/w”, “mass percent,” or “percent w/w.”

The term “crystalline” used throughout the specification and claims includes solids described as “weakly crystalline.”

The term “alkali metal salt” includes, but is not limited to, a salt where the counterion is Li, Na, K, Rb, or another Group IA counterion.

The term “alkaline earth metal salt” includes, but is not limited to, a salt where the counterion is Be, Mg, Ca, Sr, or another Group IIA counterion.

The term “divalent” is used to describe the oxidation state of a metal ion and includes, but is not limited to, Mg²⁺, Ca²⁺, Zn²⁺, Be²⁺, and Sr²⁺.

The term “E681010” is used to describe an omega-3 oil which has a composition comprising 67.8 percent EPA (mg/g), 9.9 percent DHA (mg/g), and about 9.6 percent other omega-3 oils (mg/g), where the EPA, DHA, and other omega-3 oils are ethyl esters.

The terms “chemically stable” or “chemical stability” refer to a liquid formulation where there is a ≦3.0 percent loss of API potency (recovered API content) after 2 years at 25 degrees C.

“Surfactants” refer to a surface active compound which can alter the surface tension of a liquid in which it is dissolved and includes, but is not limited to, polyoxyl 35 castor oil and sorbitan monolaurate.

The term “aqueous solubility” refers to the solubility as measured in deionized water at about 25 degrees C., unless otherwise specified.

“Statin” as used herein includes, but is not limited to, pravastatin, fluvastatin, atorvastatin, lovastatin, simvastatin, rosuvastatin, and cerivastatin. Statins may be in'the form of a salt, hydrate, solvate, polymorph, or a co-crystal. Statins may also be in the form of a hydrate, solvate, polymorph, or a co-crystal of a salt. Statins may also be present in the free acid or lactone form according to the present invention.

The present invention comprises a suspension of one or more salts of a statin(s) in an omega-3 oil. In one embodiment, the suspension comprises solid crystalline particles of one or more salts of a statin(s) in an omega-3 oil. In another embodiment, the suspension comprises solid amorphous particles of one or more salts of a statin(s) in an omega-3 oil. In another embodiment, the suspension comprises solid crystalline and solid amorphous particles of one or more salts of a statin(s) in an omega-3 oil. Also included in the present invention are pharmaceutical formulations comprising suspensions of one or more salts of a statin(s) in an omega-3 oil where a portion of said one or more salts of a statin(s) is solubilized in the omega-3 oil or in additional component(s) of the formulation. For example, in another embodiment, the present invention provides a pharmaceutical formulation comprising an omega-3 oil and one or more salts of a statin(s), wherein about 1.00, 2.00, 3.00, 4.00, 5.00, 6.00, 7.00, 8.00, 9.00, 10.00, 11.00, 12.00, 13.00, 14.00, or 15.00 percent statin(s) by weight is/are in solution while the remaining statin(s) is/are present in suspension.

In another embodiment, the present invention provides a pharmaceutical formulation comprising an omega-3 oil and one or more salts of a statin(s), wherein at least about 80 percent of the statin(s) by weight are present as solid particles in suspension. In another embodiment, the present invention provides a pharmaceutical formulation comprising an omega-3 oil and one or more salts of a statin(s), wherein at least about 85 percent of the statin(s) by weight are present as solid particles in suspension. In another embodiment, the present invention provides a pharmaceutical formulation comprising an omega-3 oil and one or more salts of a statin(s), wherein at least about 90 percent of the statin(s) by weight are present as solid particles in suspension. In another embodiment, the present invention provides a pharmaceutical formulation comprising an omega-3 oil and one or more salts of a statin(s), wherein at least about 95 percent of the statin(s) by weight are present as solid particles in suspension. In another embodiment, the present invention provides a pharmaceutical formulation comprising an omega-3 oil and one or more salts of a statin(s), wherein at least about 99 percent of the statin(s) by weight are present as solid particles in suspension.

The purity of omega-3 oil is an important aspect of the present invention. Oil purity is defined as a percentage (e.g., by volume or by weight) of one component of the oil with respect to the entire oil composition. For example, an ester oil with a purity of 95 percent by weight comprises at least 95 percent w/w esters. The remaining percentage may comprise free acids, mono- di- and/or triglycerides, or other components. As another example, an omega-3 ester oil with a purity of 90 percent by weight comprises at least 90 percent w/w omega-3 esters and the remaining percentage can comprise any one or more of other oil components. A mixture of species of one component (e.g., C₈ and C₁₀ esters) need not be discerned in the determination of purity. However, a distinction of specific species within a component (e.g., C₈ and C₁₀ esters) can also be included in specific embodiments of the present invention.

According to the present invention, omega-3 oils with a purity greater than about 85 percent, 90 percent, 91 percent, 92 percent, 93 percent, 94 percent, 95 percent, 96 percent, 97 percent, 98 percent, 99 percent or more can be used, for example, in a pharmaceutical formulation. Omega-3 oils, specifically with a high purity of omega-3 esters, can be used. According to the present invention, omega-3 oils with a high purity comprise greater than about 85 percent, 90 percent, 91 percent, 92 percent, 93 percent, 94 percent, 95 percent, 96 percent, 97 percent, 98 percent, 99 percent or more of one component by weight or by volume. Omega-3 esters include, but are not limited to, EPA and DHA. Omega-3 esters also include omega-3 ethyl esters.

Oils containing pure and substantially pure alkyl esters are included in the present invention. However, mixtures of omega-3 alkyl esters with other components of omega-3 oil (e.g., fatty acids, triglycerides) are also included, according to the present invention.

In another embodiment, the purity of omega-3 esters or omega-3 alkyl esters is at least about 50 percent by weight, at least about 60 percent by weight, at least about 70 percent by weight, at least about 75 percent by weight, at least about 80 percent by weight, or at least about 85 percent by weight. In another embodiment, the purity of omega-3 esters or omega-3 alkyl esters is about 25, 30, 35, 40, 45, 50, 55, 60, 65, 70, 75, 80, 85, 90, 95, 99 percent or more by weight. In another embodiment, the purity of omega-3 esters or omega-3 alkyl esters is between about 25 and about 100 percent by weight, between about 40 and about 100 percent by weight, between about 50 and about 100 percent by weight, between about 60 and about 100 percent by weight, between about 70 and about 100 percent by weight, between about 75 and about 100 percent by weight, between about 75 and about 95 percent by weight, between about 75 and about 90 percent by weight, or between about 80 and about 85 percent by weight. In another embodiment, the purity of omega-3 esters or omega-3 alkyl esters is about 100 percent by weight, about 99 percent by weight, about 96 percent by weight, about 92 percent by weight, about 90 percent by weight, about 85 percent by weight, about 80 percent by weight, about 75 percent by weight, about 70 percent by weight, about 65 percent by weight, about 60 percent by weight, about 55 percent by weight, or about 50 percent by weight.

In another embodiment, the oil composition comprising EPA and DHA is at least about 50 percent by weight, at least about 60 percent by weight, at least about 70 percent by weight, at least about 75 percent by weight, at least about 80 percent by weight, or at least about 84 percent by weight. In another embodiment, the oil composition comprising EPA and DHA is about 25, 30, 35, 40, 45, 50, 55, 60, 65, 70, 75, 80, 85, 90, or 95 percent by weight. In another embodiment, the oil composition comprising EPA and DMA is between about 25 and about 95 percent by weight, between about 40 and about 95 percent by weight, between about 50 and about 95 percent by weight, between about 60 and about 95 percent by weight, between about 70 and about 95 percent by weight, between about 75 and about 95 percent by weight, between about 75 and about 90 percent by weight, between about 75 and about 85 percent by weight, or between about 80 and about 85 percent by weight. In another embodiment, the oil composition comprising EPA and DHA is about 99 percent by weight, about 96 percent by weight, about 92 percent by weight, about 90 percent by weight, about 84 percent by weight, about 80 percent by weight, about 75 percent by weight, about 70 percent by weight, about 65 percent by weight, about 60 percent by weight, about 55 percent by weight, or about 50 percent by weight.

In another embodiment, the omega-3 ester or omega-3 alkyl ester has about a 23:19 ratio of EPA:DHA, about a 75:11 ratio of EPA:DHA, about a 95: I ratio of EPA:DHA, about a 9:2 ratio of EPA:DHA, about a 10:1 ratio of EPA:DHA, about a 5:1 ratio of EPA:DHA, about a 3:1 ratio of EPA:DHA, about a 2:1 ratio of EPA:DHA, about a 1:1 ratio of EPA:DHA, about a 1:2 ratio of EPA:DHA, about a 1:3 ratio of EPA:DHA, or about a 1:5 ratio of EPA:DHA. In another embodiment, the omega-3 ester or omega-3 alkyl ester has about a 95:1 ratio of EPA:DHA, about a 75:1 ratio of EPA:DHA, about a 50:1 ratio of EPA:DHA, about a 25:1 ratio of EPA:DHA, about a 20:1 ratio of EPA:DHA, about a 15:1 ratio of EPA:DHA, about a 10:1 ratio of EPA:DFIA, about a 7.5:1 ratio of EPA:DHA, about a 5:1 ratio of EPA:DHA, about a 4:1 ratio of EPA:DHA, about a 3:1 ratio of EPA:DHA, about a 2:1 ratio of EPA:DLIA, about a 1.5:1 ratio of EPA:DHA, about a 1:1 ratio of EPA:DHA, about a 1:1.5 ratio of EPA:DHA, about a 1:2 ratio of EPA:DHA, about a 1:3.ratio of EPA:DHA, or about a 1:5 ratio of EPA:DHA. In another embodiment, the omega-3 ester or omega-3 alkyl ester has from about a 95:1 ratio to about a 1:5 ratio of EPA:DHA, from about a 50:1 ratio to about a 1:1 ratio of EPA:DHA, from about a 25:1 ratio to about a 1:1 ratio of EPA:DHA, from about a 10:1 ratio to about a 1:1 ratio of EPA:DHA, from about a 5:1 ratio to about a 1:1 ratio of EPA:DHA, from about a 3:1 ratio to about a 1:1 ratio of EPA:DHA, from about a 2:1 ratio to about a 1:1 ratio of EPA:DHA, or from about a 1.5:1 ratio to about a 1:1 ratio of EPA:DHA. In another embodiment, the omega-3 ester or omega-3 alkyl ester has at least about a 1:5 ratio of EPA:DHA, at least about a 1:1 ratio of EPA:DHA, at least about a 1.5:1 ratio of EPA:DHA, at least about a 2:1 ratio of EPA:DHA, at least about a 3:1 ratio of EPA:DHA, at least about a 5:1 ratio of EPA:DHA, or at least about a 10:1 ratio of EPA:DHA.

In another embodiment, the present invention provides a salt of a statin. In a specific embodiment, a calcium salt of pravastatin is provided. In another specific embodiment, a magnesium salt of pravastatin is provided. In another specific embodiment, a zinc salt of pravastatin is provided. In another specific embodiment, a calcium salt of fluvastatin is provided. In another embodiment, a divalent salt of a statin is provided. In another embodiment, the salt of a statin is amorphous. In another embodiment, the salt of a statin is crystalline.

In another embodiment, a pharmaceutical formulation or a medicament comprising a salt of a statin is provided.

In another embodiment, the present invention provides a method for preparing a salt of a statin.

In another embodiment, a method for preparing a salt of a statin comprises:
(a) combining a statin and a salt in solution;
(b) initiating precipitation of a salt of said statin; and
(c) collecting said salt of said statin.

In another embodiment, the statin in step (a) can be a salt. For example, the statin in step (a) can be an alkali metal salt of a statin, such as, but not limited to, pravastatin sodium salt or fluvastatin sodium salt. In another embodiment, the salt in step (a) can be an alkaline earth metal salt. For example, the salt in step (a) can be a calcium or a magnesium salt, such as, but not limited to, calcium acetate or calcium chloride.

In another embodiment, initiating precipitation in step (b) can be completed by cooling the solution, evaporating the solution or a portion thereof, or one or more of techniques known to one skilled in the art.

In another embodiment, collecting the salt in step (c) can be completed via filtration, decanting, or any one or more of techniques known to one skilled in the art.

The statin salt forms described herein can also take the form of a polymorph, co-crystal, hydrate, or solvate.

In another embodiment, a pharmaceutical formulation or medicament of the present invention comprises about 500, 600, 700, 800, 900, 1000, 1100, 1200, 1300, 1400, or 1500 mg of omega-3 ester and about 1, 2, 3, 4, 5, 10, 15, 20, 25, 30, 35, 40, 45, 50, 55, 60, 65, 70, 75, or 80 mg of a salt of pravastatin.

In another embodiment, a pharmaceutical formulation or medicament of the present invention comprises about 500, 600, 700, 800, 900, 1000, 1100, 1200, 1300, 1400, or 1500 mg of omega-3 ester and about 1, 2, 3, 4, 5, 10, 15, 20, 25, 30, 35, 40, 45, 50, 55, 60, 65, 70, 75, 80, 90, 100, 110, 120, 130, 140, 150, or 160 mg of a salt of fluvastatin.

In another embodiment, a pharmaceutical formulation or medicament of the present invention comprises about 500, 600, 700, 800, 900, 1000, 1100, 1200, 1300, 1400, or 1500 mg of omega-3 ethyl ester and about 1, 2, 3, 4, 5, 10, 15, 20, 25, 30, 35, 40, 45, 50, 55, 60, 65, 70, 75, 80, 90, 100, 110, 120, 130, 140, 150, or 160 mg of a salt of a statin(s).

According to the present invention, the mass of a salt of a statin is measured with respect to the mass of the free form. For example, an 80 mg amount of a salt of a statin refers to 80 mg of free form statin, without the mass of the cation being included.

In another embodiment, a thickener, such as, but not limited to, calcium carbonate or silicon dioxide, can be added to a pharmaceutical formulation according to the present invention.

In another embodiment, a pharmaceutical formulation or medicament of the present invention can be stored for up to 8 weeks at about 25 degrees C. with no detectable degradation of the statin(s). In another embodiment, a pharmaceutical formulation of the present invention can be stored for up to 12 weeks at about 25 degrees C. with no detectable degradation of the statin(s). In another embodiment, a pharmaceutical formulation of the present invention can be stored for up to 16 weeks at about 25 degrees C. with no detectable degradation of the statin(s). In another embodiment, a pharmaceutical formulation of the present invention can be stored for up to 26 weeks at about 25 degrees C. with no detectable degradation of the statin(s).

In another embodiment, pravastatin calcium salt exhibits an unexpectedly high stability in a suspension of omega-3 oil relative to other pravastatin salts. Pravastatin magnesium and zinc salts and fluvastatin calcium salt are also non-limiting statins according to the present invention.

In another embodiment, pravastatin zinc salt exhibits an unexpectedly high stability in a suspension of omega-3 oil and an alcohol relative to other pravastatin salts, as shown in the Exemplification. Surprisingly, a suspension of pravastatin zinc salt displays a higher stability in omega-3 oil than other pravastatin salts, such as the sodium, calcium, and potassium salts.

In another embodiment, a method of preventing, reducing, and/or treating hypercholesterolemia, atherosclerosis, hyperlipidemia, mixed dyslipidemia, cardiovascular events and disease including coronary events and cerebrovascular events, and coronary artery disease and/or cerebrovascular disease is provided by administering an effective amount of a pharmaceutical formulation of the present invention to a mammal in need of such prevention, reduction, and/or treatment. In another embodiment, the mammal is a human.

In another embodiment, the present invention includes a salt of a statin with an aqueous solubility less than about 200.00 mg/mL. For example, less than about 200.00, 190.00, 180.00, 170.00, 160.00, 150.00, 140.00, 130.00, 120.00, 110.00, 100.00, 90.00, 80.00, 75.00, 70.00, 65.00, 60.00, 55.00, 50.00, 45.00, 40.00, 35.00, or less than about 30.00 mg/mL. In another embodiment, the present invention includes a salt of a statin with an aqueous solubility less than about 25.00 mg/mL or an aqueous solubility ranging between about 0.10 mg/mL and about 25 mg/mL. In another embodiment, the present invention includes a salt of pravastatin with an aqueous solubility less than about 200.00 mg/mL. For example, less than about 200.00, 190.00, 180.00, 170.00, 160.00, 150.00, 140.00, 130.00, 120.00, 110.00, 100.00, 90.00, 80.00, 75.00, 70.00, 65.00, 60.00, 55.00, 50.00, 45.00, 40.00, 35.00, or less than about 30.00 mg/mL. In another embodiment, a pravastatin salt of the present invention has an aqueous solubility less than about 25.00 mg/mL or an aqueous solubility ranging between 0.10 mg/mL and about 25 mg/mL. In another embodiment, the present invention includes a statin salt or a pravastatin salt with an aqueous solubility less than (or less than about) 25.00, 24.00, 23.00, 22.00, 21.00, 20.00, 19.00, 18.00, 17.00, 16.00, 15.00, 14.00, 13.00, 12.00, 11.00, 10.00, 9.00, 8.00, 7.00, 6.00, 5.00, 4.00, 3.00, 2.00, 1.00, 0.90, 0.80, 0.70, 0.60, 0.50, 0.40, 0.30, 0.20, or 0.10 mg/mL (these solubility values are to be understood as including, and providing written support for any fractional solubility in intervals of 0.01 mg/mL and such solubilities have not been included herein for the sake of brevity and to refrain from unduly lengthening the specification). Insoluble salts (salts having a solubility of 0.00 mg/mL) are not included in the scope of the invention. The aforementioned range of aqueous solubilities from about 0.10 mg/mL to about 25.00 mg/mL is to be taken as including, and providing written description and support for, any fractional solubility, in intervals of 0.01 mg/mL, between about 0.10 mg/mL and about 25.00 mg/mL. Aqueous solubilities for the pravastatin salts of the invention can also be described as having an aqueous solubility of less than (or less than about) X.YZ mg/mL, where X is 1, 2, 3, 4, 5, 6, 7, 8, 9, 10, 11, 12, 13, 14, 15, 16, 17, 18, 19, 20, 21, 22, 23, 24 or 25, Y is 0, 1, 2, 3, 4, 5, 6, 7, 8 or 9, and Z is 0, 1, 2, 3, 4, 5, 6, 7, 8 or 9 (provided that when X is 0, Y and Z cannot both be 0 [i.e., X, Y and Z cannot each, independently, be 0])

In another embodiment, the present invention includes a salt of fluvastatin with an aqueous solubility less than about 200.00 mg/mL. For example, less than about 200.00, 190.00, 180.00, 170.00, 160.00, 150.00, 140.00, 130.00, 120.00, 110.00, 100.00, 90.00, 80.00, 75.00, 70.00, 65.00, 60.00, 55.00, 50.00, 45.00, 40.00, 35.00, or less than about 30.00 mg/mL. In another embodiment, the present invention includes a salt of fluvastatin with an aqueous solubility less than about 25.00 mg/mL or an aqueous solubility ranging between 0.10 mg/mL and about 25 mg/mL. In another embodiment, the present invention includes a fluvastatin salt with an aqueous solubility less than about 25.00, 24.00, 23.00, 22.00, 21.00, 20.00, 19.00, 18.00, 17.00, 16.00, 15.00, 14.00, 13,00, 12.00, 11.00, 10.00, 9.00, 8.00, 7.00, 6.00, 5.00, 4.00, 3.00, 2.00, 1.00, 0.90, 0.80, 0.70, 0.60, 0.50, 0.40, 0.30, 0.20, or 0.10 mg/mL (these solubility values are to be understood as including, and providing written support for any fractional solubility in intervals of 0.01 mg/mL and such solubilities have not been included herein for the sake of brevity and to refrain from unduly lengthening the specification). Insoluble salts (salts having a solubility of 0.00 mg/mL) are not included in the scope of the invention. The aforementioned range of aqueous solubilities from about 0.10 mg/mL and about 25.00 mg/mL is to be taken as including, and providing written description and support for, any fractional percentage, in intervals of 0.01 mg/mL, between about 0.10 mg/mL and about 25.00 mg/mL. Aqueous solubilities for the fluvastatin salts of the invention can also be described as having an aqueous solubility of less than (or less than about) X.YZ mg/mL, where X is 1, 2, 3, 4, 5, 6, 7, 8, 9, 10, 11, 12, 13, 14, 15, 16, 17, 18, 19, 20, 21, 22, 23, 24 or 25, Y is 0, 1, 2, 3, 4, 5, 6, 7, 8 or 9, and Z is 0, 1, 2, 3, 4, 5, 6, 7, 8 or 9 (provided that when X is 0, Y and Z cannot both be 0 [i.e., X, Y and Z cannot each, independently, be 0]).

In another embodiment, the present invention provides a pharmaceutical formulation of a salt of a statin as described above where the salt of a statin has an aqueous solubility less than about 25 mg/mL.

In another embodiment, a pharmaceutical formulation or medicament of the present invention comprises about 500, 600, 700, 800, 900, 1000, 1100, 1200, 1300, 1400, or 1500 mg of omega-3 oil and about 1, 2, 3, 4, 5, 10, 15, 20, 25, 30, 35, 40, 45, 50, 55, 60, 65, 70, 75, 80, 90, 100, 110, 120, 130, 140, 150, or 160 mg of a salt of a statin(s), where the salt has an aqueous solubility less than about 200 mg/mL. In another embodiment, a pharmaceutical formulation or medicament of the present invention comprises about 500, 600, 700, 800, 900, 1000, 1100, 1200, 1300, 1400, or 1500 mg of omega-3 oil and about 1, 2, 3, 4, 5, 10, 15, 20, 25, 30, 35, 40, 45, 50, 55, 60, 65, 70, 75, 80, 90, 100, 110, 120, 130, 140, 150, or 160 mg of a salt of a statin(s), where the salt has an aqueous solubility less than about 50 mg/mL. In another embodiment, a pharmaceutical formulation or medicament of the present invention comprises about 500, 600, 700, 800, 900, 1000, 1100, 1200, 1300, 1400, or 1500 mg of omega-3 oil and about 1, 2, 3, 4, 5, 10, 15, 20, 25, 30, 35, 40, 45, 50, 55, 60, 65, 70, 75, 80, 90, 100, 110, 120, 130, 140, 150, or 160 mg of a salt of a statin(s), where the salt has an aqueous solubility less than about 25 mg/mL. In a specific embodiment, a pharmaceutical formulation or medicament of the present invention comprises about 500, 600, 700, 800, 900, 1000, 1100, 1200, 1300, 1400, or 1500 mg of omega-3 oil and about 1, 2, 3, 4, 5, 10, 15, 20, 25, 30, 35, 40, 45, 50, 55, 60, 65, 70, 75, 80, 90, 100, 110, 120, 130, 140, 150, or 160 mg of a salt of a statin(s), where the salt has an aqueous solubility of about 15-17 mg/mL. In another embodiment, a pharmaceutical formulation or medicament of the present invention comprises about 500, 600, 700, 800, 900, 1000, 1100, 1200, 1300, 1400, or 1500 mg of omega-3 oil and about 1, 2, 3, 4, 5, 10, 15, 20, 25, 30, 35, 40, 45, 50, 55, 60, 65, 70, 75, 80, 90, 100, 110, 120, 130, 140, 150, or 160 mg of a salt of a statin(s), where the salt has an aqueous solubility of about 0.5 mg/mL. In another embodiment, a pharmaceutical formulation or medicament of the present invention comprises about 500, 600, 700, 800, 900, 1000, 1100, 1200, 1300, 1400, or 1500 mg of omega-3 oil and about 1, 2, 3, 4, 5, 10, 15, 20, 25, 30, 35, 40, 45, 50, 55, 60, 65, 70, 75, 80, 90, 100, 110, 120, 130, 140, 150, or 160 mg of a salt of a statin(s), where the salt has an aqueous solubility of about 0.3 mg/mL.

A therapeutically acceptable daily dosage of omega-3 oil has been recommended or considered via several national and international groups including, but not limited to, the American Heart Association (AHA) and the International Society for the Study of Fatty Acids and Lipids (ISSFAL). Table 1 includes daily dosage amounts of omega-3 as considered/recommended via several organizations.

TABLE 1
Daily dosages of omega-3

| Omega-3 dose | |
|---|---|
| (grams)/day | Comment |
| 0.65 | ISSFAL consideration (1999) |
| 1.0 | AHA recommended (2000, 2004) |
| 1.8 | Omancor ® dose |
| 3.0 | FDA limit on daily consumption, general population |
| 3.6 | Omacor ® dose |

Pharmaceutical dosage forms of one or more statin salts of the present invention can be administered in several ways including, but not limited to, oral administration. Oral pharmaceutical compositions and dosage forms are exemplary dosage forms. Optionally, the oral dosage form is a solid dosage form, such as a tablet, a caplet, a hard gelatin capsule, a starch capsule, a hydroxypropyl methylcellulose (HPMC) capsule, or a soft elastic gelatin capsule. Liquid dosage forms may also he provided by the present invention, including such non-limiting examples as a suspension, solution, syrup, or emulsion.

A statin salt of the present invention can be administered by controlled or delayed release means. Controlled release pharmaceutical products generally have a common goal of improving drug therapy over that achieved by their non-controlled release counterparts. Ideally, the use of an optimally designed controlled release preparation in medical treatment is characterized by a minimum of API (active pharmaceutical ingredient) substance being employed to cure or control the condition in a minimum amount of time. Advantages of controlled release pharmaceutical compositions generally include: 1) extended activity of the API; 2) reduced dosage frequency; 3) increased patient compliance; 4) usage of less total API; 5) reduction in local or systemic side effects; 6) minimization of API accumulation; 7) reduction in blood level fluctuations; 8) improvement in efficacy of treatment; 9) reduction of potentiation or loss of API activity; and 10) improvement in speed of control of diseases or conditions. (Kim, Chemg-ju, Controlled Release Dosage Form Design, 2 Technomic Publishing, Lancaster, Pa.: 2000).

Typical daily dosage forms of the invention comprise a statin salt, in an amount of from about 5.0 mg to about 160.0 mg, from about 10.0 mg to 80.0 mg, or from about 10.0 mg to about 40.0 mg. In a particular embodiment, the statin salt for use in such a composition is pravastatin calcium salt, pravastatin magnesium salt, pravastatin zinc salt, or fluvastatin calcium salt. The dosage amounts described herein are expressed in amounts of the free form statin and do not include the weight of a counterion (e.g., Ca²⁺, Mg²⁺, Zn²⁺) or any water or solvent molecules.

In another embodiment of the invention, a pharmaceutical composition comprising a statin salt is administered orally as needed in an amount of from about 5.0 mg to about 160.0 mg, from about 10.0 mg to about 80.0 mg, from about 10.0 mg to about 40.0 mg, or from about 20.0 mg to about 40.0 mg statin. For example, about 10.0 mg, about 20.0 mg, about 40.0 mg, or about 80.0 mg. The dosage amounts can be administered in single or divided doses. In another embodiment, a daily dose of a pharmaceutical composition comprising a statin salt comprises up to about 160.0 mg statin. In other embodiments, the present invention is directed to compositions comprising a statin salt as described herein and one or more diluents, carriers, and/or excipients suitable for the administration to a mammal for the treatment or prevention of one or more of the conditions described herein.

The statin salts of the present invention may also be used to prepare pharmaceutical dosage forms other than the oral dosage forms described above, such as topical dosage forms, parenteral dosage forms, transdermal dosage forms, and mucosal dosage forms. For example, such forms include creams, lotions, solutions, suspensions, emulsions, ointments, powders, patches, suppositories, and the like.

The statin salt forms of the present invention can be characterized, e.g., by TGA, DSC, DVS, single crystal x-ray diffractometer data, or by any one, any two, any three, any four, any five, any six, any seven, any eight, any nine, any ten, or any single integer number of PXRD 2-theta angle peaks, or by any combination of the data acquired from the analytical techniques described herein.

Although the invention has been described with respect to various embodiments, it should be realized this invention is also capable of a wide variety of further and other embodiments within the spirit and scope of the appended claims.

### EXEMPLIFICATION

### Materials and Methods

Differential scanning calorimetric (DSC) analysis of the samples was performed using a Q 1000 Differential Scanning calorimeter (TA Instruments, New Castle, Del., U.S.A.), which uses Advantage for QW-Series, version 1.0.0.78, Thermal Advantage Release 2.0 (2001 TA Instruments-Water LLC). In addition, the analysis software used was Universal Analysis 2000 for Windows 95/98/2000/NT, version 3.1 E; Build 3.1.0.40 (2001 TA Instruments-Water LLC).

For the DSC analysis, the purge gas used was dry nitrogen, the reference material was an empty aluminum pan that was crimped, and the sample purge was 50 mL/minute.

DSC analysis of the sample was performed by placing the modafinil sample in an aluminum pan with a crimped pan closure. The starting temperature was typically 20 degrees C. with a heating rate of 10 degrees C./minute, and the ending temperature was 200 degrees C. All reported DSC transitions represent the temperature of endothermic or exothermic transition at their respective peaks with an error of +/−2 degrees C., unless otherwise indicated.

Thermogravimetric analysis (TGA) of samples was performed using a Q500 Thermogravimetric Analyzer (TA Instruments, New Castle, Del., U.S.A.), which uses Advantage for QW-Series, version 1.0.0.78, Thermal Advantage Release 2.0 (2001 TA Instruments-Water LLC). In addition, the analysis software used was Universal Analysis 2000 for Windows 95/98/2000/NT, version 3.IE; Build 3.1.0.40 (2001 TA Instruments-Water LLC).

For the TGA experiments, the purge gas used was dry nitrogen, the balance purge was 40 mL/minute N₂, and the sample purge was 60 mL/minute N₂.

TGA was performed on the sample by placing the modafinil sample in a platinum pan. The starting temperature was typically 20 degrees C. with a heating rate of 10 degrees C./minute, and the ending temperature was 300 degrees C.

A powder X-ray diffraction (PXRD) pattern for some samples was obtained using a D/Max Rapid, Contact (Rigaku/MSC, The Woodlands, Tex., U.S.A.), which uses as its control software RINT Rapid Control Software, Rigaku Rapid/XRD, version 1.0.0 (1999 Rigaku Co.). In addition, the analysis software used were RINT Rapid display software, version 1.18 (Rigaku/MSC), and JADE XRD Pattern Processing, versions 5.0 and 6.0 ((1995-2002, Materials Data, Inc.).

For the PXRD analysis, the acquisition parameters were as follows: source was Cu with a K line at 1.5406 A; x-y stage was manual; collimator size was 0.3 mm; capillary tube (Charles Supper Company, Natick, Mass., U.S.A.) was 0.3 mm ID; reflection mode was used; the power to the X-ray tube was 46 kV; the current to the X-ray tube was 40 mA; the omega-axis was oscillating in a range of 0-5 degrees at a speed of 1 degree/minute; the phi-axis was spinning at an angle of 360 degrees at a speed of 2 degrees/second; 0.3 mm collimator; the collection time was 60 minutes; the temperature was room temperature; and the heater was not used. The sample was presented to the X-ray source in a boron rich glass capillary.

In addition, the analysis parameters were as follows: the integration 2-theta range was 2-60 degrees; the integration chi range was 0-360 degrees; the number of chi segments was 1; the step size used was 0.02; the integration utility was cylint; normalization was used; dark counts were 8; omega offset was 180; and chi and phi offsets were 0.

PXRD diffractograms were also acquired via the Bruker AXS D8 Discover X-ray Diffractometer. This instrument was equipped with GADDS™ (General Area Diffraction Detection System), a Bruker AXS FH-STAR Area Detector at a distance of 15.05 cm as per system calibration, a copper source (Cu/K_{α} 1.54056 angstroms), automated x-y-z stage, and 0.5 mm collimator. The sample was compacted into pellet form and mounted on the x-y-z stage. A diffractogram was acquired under ambient conditions at a powder setting of 40 kV and 40 mA in reflection mode while the sample remained stationary. The exposure time was varied and specified for each sample. The diffractogram obtained underwent a spatial remapping procedure to account for the geometrical pincushion distortion of the area detector then integrated along chi from −118.8 to −61.8 degrees and 2-theta 2.1-37 degrees at a step size of 0.02 degrees with normalization set to bin normalize.

The relative intensity of peaks in a diffractogram is not necessarily a limitation of the PXRD pattern because peak intensity can vary from sample to sample, e.g., due to crystalline impurities. Further, the angles of each peak can vary by about +/−0.1 degrees, or by about +/−0.05. The entire pattern or most of the pattern peaks may also shift by about +/−0.1 degrees to about +/−0.2 degrees due to differences in calibration, settings, and other variations from instrument to instrument and from operator to operator. All reported PXRD peaks in the Figures, Examples, and elsewhere herein are reported with an error of about ±0.1 degrees 2-theta. Unless otherwise noted, all diffractograms are obtained at about room temperature (about 24 degrees C. to about 25 degrees C.).

For PXRD, IR, and Raman data herein, including description and Figures, each composition of the present invention may be characterized by any one, any two, any three, any four, any five, any six, any seven, or any eight or more of the peaks listed (e.g., degrees 2-theta, cm⁻¹. Any one, two, three, four, five, or six DSC transitions can also be used to characterize the compositions of the present invention. The different combinations of the PXRD, IR, or Raman peaks and the DSC transitions can also be used to characterize the compositions.

### SOLUBILITY MEASUREMENTS VIA ULTRAVIOLET (UV) ABSORPTION

A calibration curve was constructed by preparing known concentrations of API (active pharmaceutical ingredient) in absolute ethanol in volumetric flasks. At each concentration, 200 microliters of the solution was transferred into a 96-well clear bottom UV plate. The sample absorbance was measured at 280 nm (unless otherwise noted) in a UV spectrophotometer. It was found that the absorbance vs. concentration correlation was linear to at least 100 micrograms/mL.

To measure the API concentration in the sample, a small aliquot was taken and diluted (typically 2000-fold) with absolute ethanol in a volumetric flask to a final approximate concentration of less than 100 micrograms/mL. The absorbance at 280 nm (unless otherwise noted) is measured and the solubility is calculated based on the calibration curve. The solubility of several statin salts were measured using the above described technique at a temperature of 20-25 degrees C.

### Example 1 A

### Pravastatin Calcium Salt

To a solution of pravastatin Na salt (1.470 g; 3.292 mmol) in water (15.0 mL) was added a solution of calcium acetate (268 mg; 1.70 mmol) also in water (5.0 mL). The resulting solution was concentrated (through evaporation of water via a stream of nitrogen gas) to about 15 mL and cooled to 0 degrees C. A white solid precipitated and was collected via filtration. The filtrate was cooled again to 0 degrees C. which yielded further precipitation. After filtration, the solids were combined and dried in a dessicator. The resultant solid was determined to be pravastatin calcium salt. The resultant salt was a 2:1 pravastatin to calcium salt.

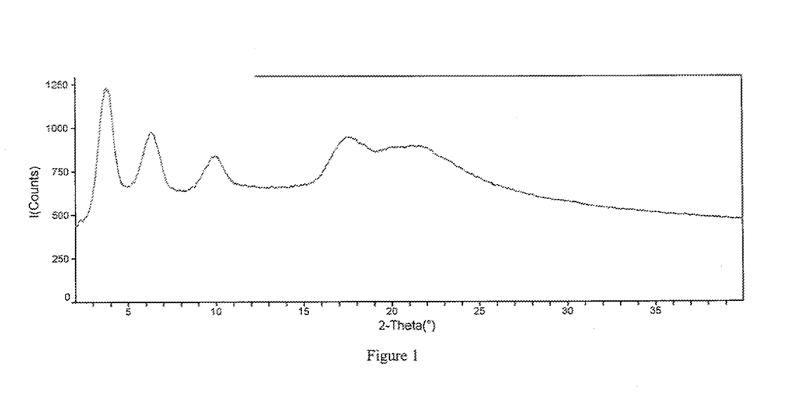

Crystals representative of those obtained by completing the method above were characterized using PXRD, TGA, IR spectroscopy, and dynamic vapor sorption (DVS). FIG. 1 shows the PXRD diffractogram of the pravastatin calcium salt (Braes, data as collected). Based on the PXRD diffractogram, the pravastatin calcium salt appears to be weakly crystalline.

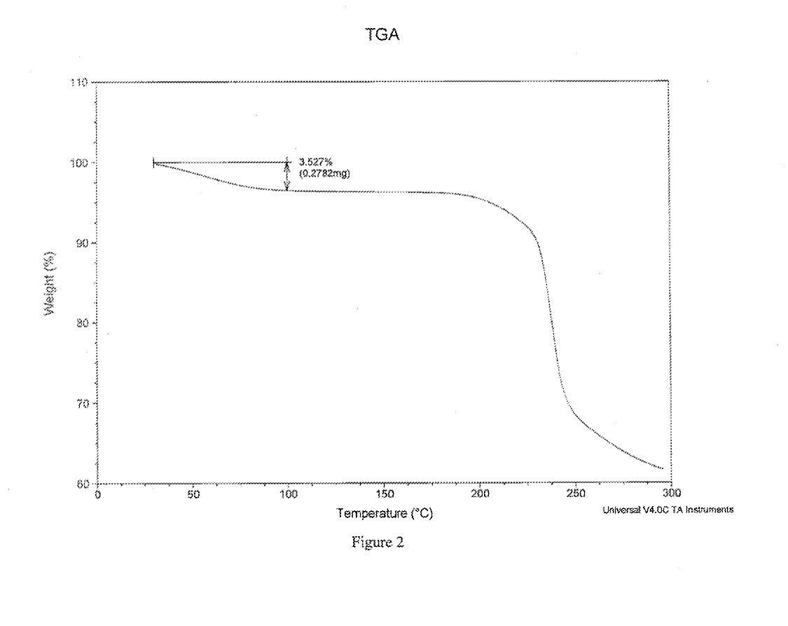

TGA of the pravastatin calcium salt showed about a 3.5 percent weight loss between about 25 degrees C. and about 100 degrees C. (See FIG. 2).

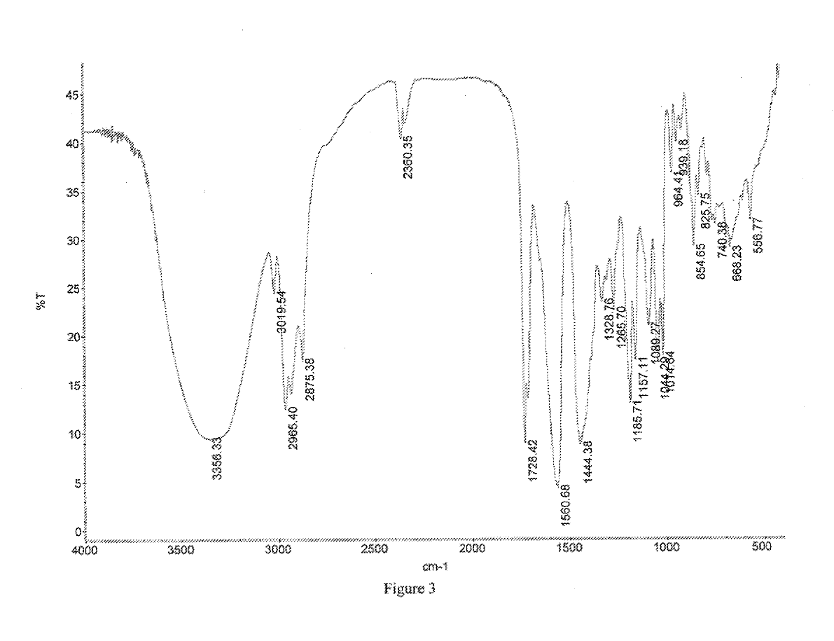

The pravastatin calcium salt exhibits an IR spectrum comprising peaks, for example, at about 2360, 1728, 1561, 1444, 1186, 855, and about 668 cm⁻¹ (See FIG. 3).

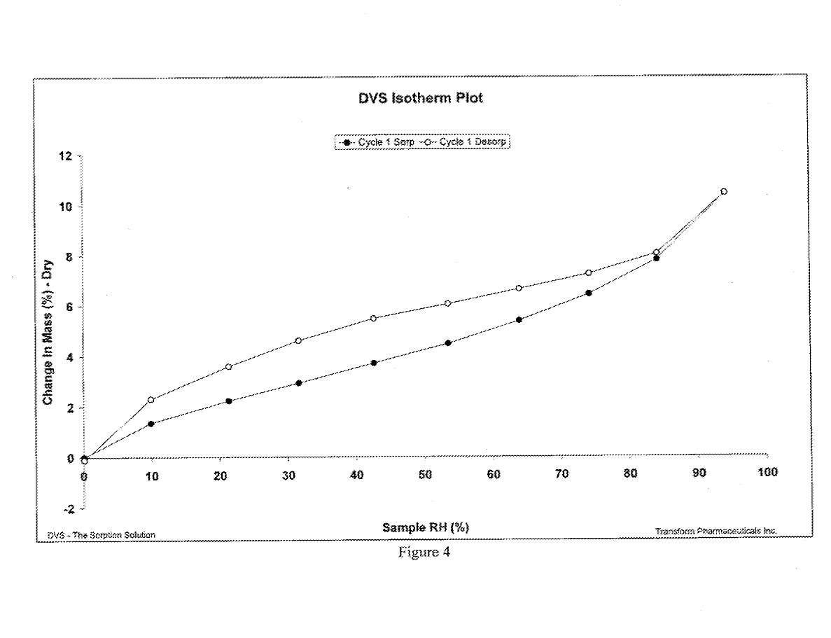

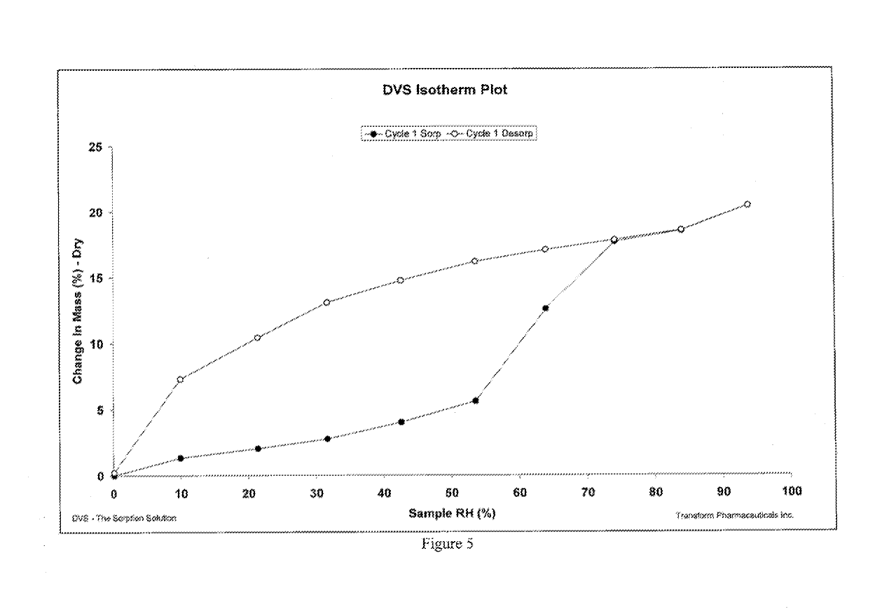

Dynamic vapor sorption (DVS) data were also acquired on both the pravastatin calcium salt and the pravastatin sodium salt. FIG. 4 shows a moisture sorp-desorp cycle of the pravastatin calcium salt. The calcium salt showed continuous water adsorption as a function of relative humidity (RH) up to about 11 percent mass gain. This is consistent with an amorphous compound. Hysteresis is observed in the desorption cycle. FIG. 5 shows a moisture sorp-desorp cycle of the pravastatin sodium salt. The sodium salt, a crystalline salt, showed a gradual increase in mass with humidity up to about 54 percent RH. Above 54 percent RH, adsorbed water increased significantly. Significant hysteresis is observed in the desorption cycle. The pravastatin sodium salt showed a greater hygroscopicity than the calcium salt.

The aqueous solubility of the calcium salt of pravastatin was determined to be about 17-20 mg/mL (via UV detection, 20-25 degrees C.). The aqueous solubility or the sodium salt of pravastatin was measured to be greater than 300 mg/mL.

### Example 1B

### Pravastatin Calcium Salt

A second method was also used to prepare pravastatin calcium salt: To a solution of pravastatin Na salt (496 mg; 1.11 mmol) in water (5.0 mL) was added a solution of calcium chloride (69 mg; 0.62 mmol) also in water (2.0 mL). The resulting solution was evaporated yielding a white solid. Pravastatin Ca salt was extracted from the solid with dry ethanol (10.0 mL) and filtered. The solution was evaporated yielding an oil which was triturated using diethyl ether (10.0 mL). The powdery white solid (100 mg) was washed with cold water (5.0 mL) and air-dried. The resultant solid was determined to be pravastatin calcium salt.

Crystals representative of those obtained by completing the method above were characterized using PXRD, TGA, IR spectroscopy, and dynamic vapor sorption (DVS). These data are discussed in Example 1A and shown in FIGS. 1-5.

### Example 2

### Fluvastatin Calcium Salt

505.9 mg (1.167 mmol) of fluvastatin Na salt was dissolved in 15 mL of water. 94.2 mg (0.595 mmol) of calcium acetate .was dissolved in 2 mL of water. A precipitate formed immediately with the addition of the calcium acetate solution to the fluvastatin Na solution. Solids were collected by filtration and dried first in a vacuum oven at 65 degrees C. for 0.5 hours and left at room temperature under nitrogen flow overnight. Dried solids were lightly ground in a mortar and pestle before characterization. The resultant solid was characterized using PXRD, DSC, TGA, Raman, and IR spectroscopy and determined to be a calcium salt of fluvastatin. The resultant salt was a 2:1 fluvastatin to calcium salt.

Solubility measurements of the sodium salt and of the calcium salt of fluvastatin were acquired in water at 23 degrees C. Solubility was measured gravimetrically in deionized water. 5.5 mg of fluvastatin sodium salt was dissolved in about 130 to 150 microliters of water, which yielded an aqueous solubility of the sodium salt of about 37 to 42 mg/mL. 5.5 mg of the calcium salt did not completely dissolve in water, even after adding up to 20 mL of water. Aqueous solubility of the calcium salt was determined to be less than or equal to about 0.275 mg/mL.

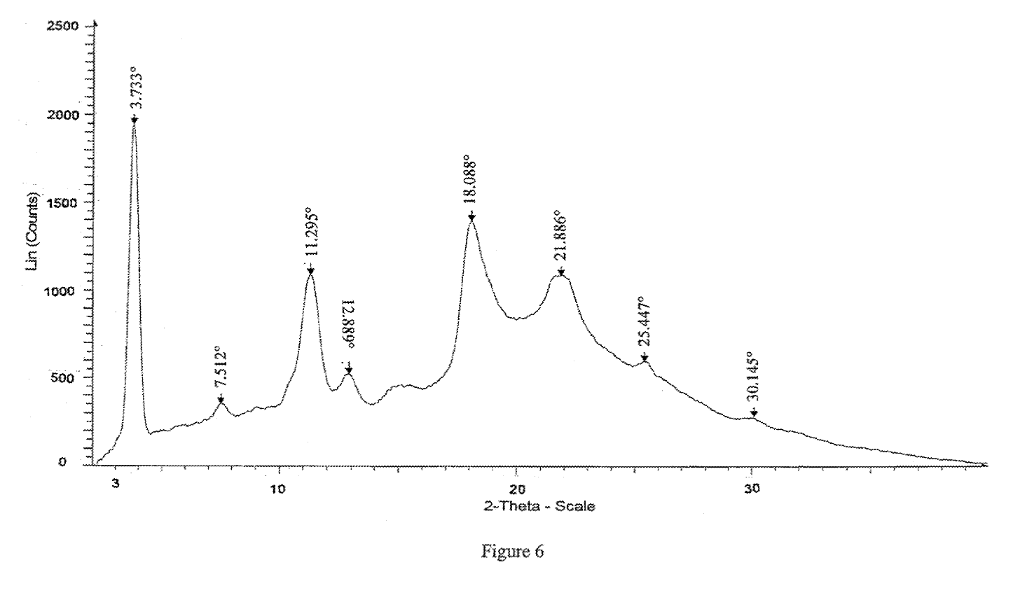

Crystals representative of those obtained by completing the method above were characterized using PXRD, DSC, TGA, Raman spectroscopy, and IR spectroscopy. The fluvastatin calcium salt exhibits a PXRD diffractogram (See FIG. 6) comprising peaks, for example, at about 3.7, 7.5, 11.3, 12.9, 18.1, 21.9, and about 25.4 degrees 2-theta (Rigaku, data as collected). Based on the PXRD diffractogram, the fluvastatin calcium salt appears to be weakly crystalline.

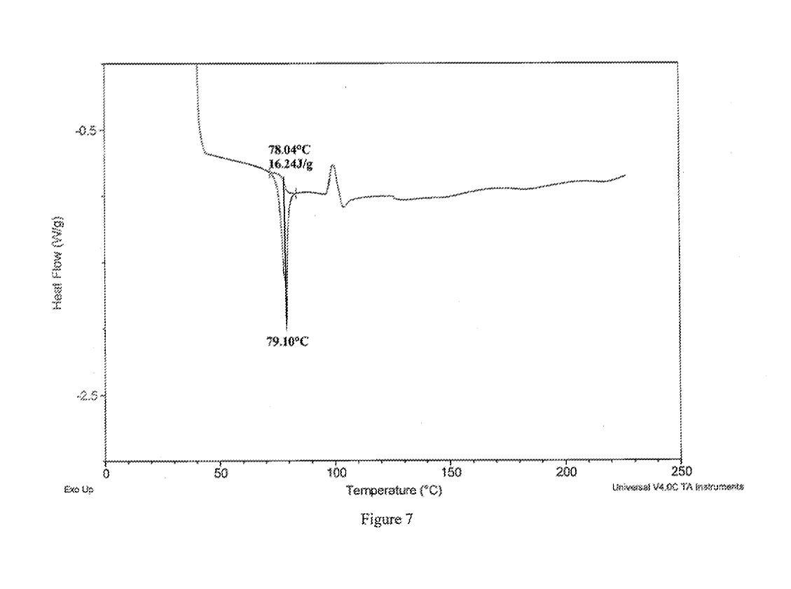

DSC was run from 25 degrees C. to 230 degrees Cat 10 degrees C./minute. DSC showed an endothermic transition at about 79 degrees C. (See FIG. 7). Note, the exotherm and small endotherm around 100 degrees C. is an artifact of the instrument and not related to the sample.

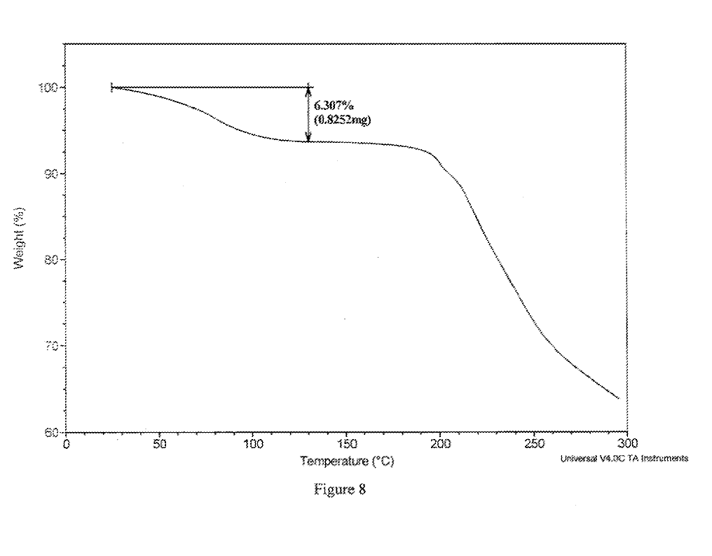

TGA (13.083 mg) was run from 25 degrees C. to 300 degrees Cat 10 degrees C./minute. TGA showed a 6.3 percent weight loss between 25 degrees C. and 130 degrees C., which may correspond to about 1.5 equivalents of water (See FIG. 8).

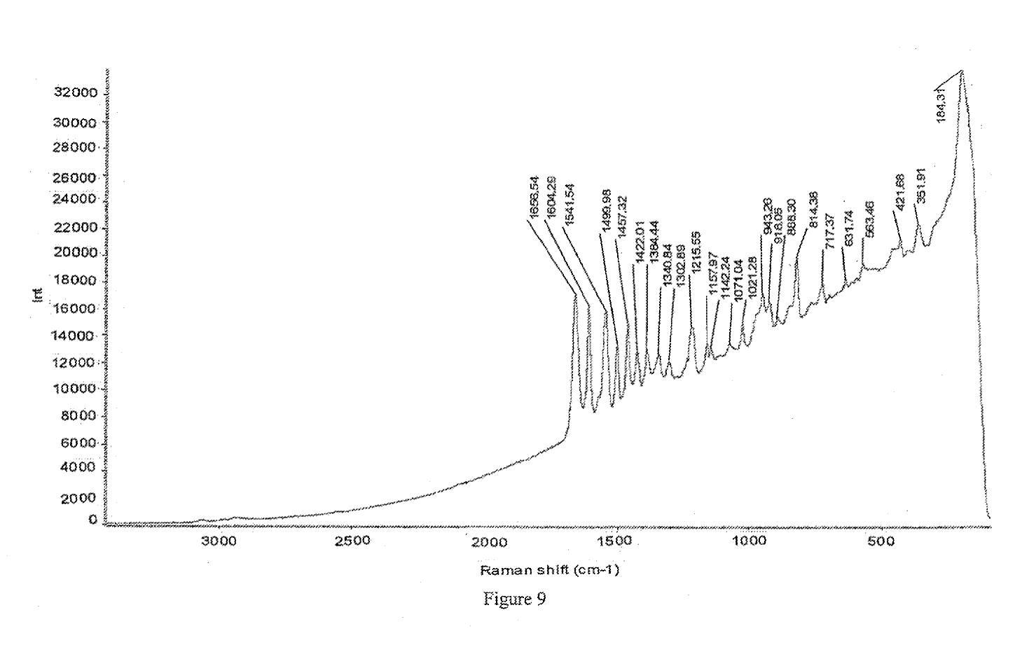

The fluvastatin calcium salt exhibits a Raman spectrum (See FIG. 9) comprising peaks, for example, at about 1657, 1604, 1542, 1500, 1457, 1216, 814, and about 352 cm⁻¹.

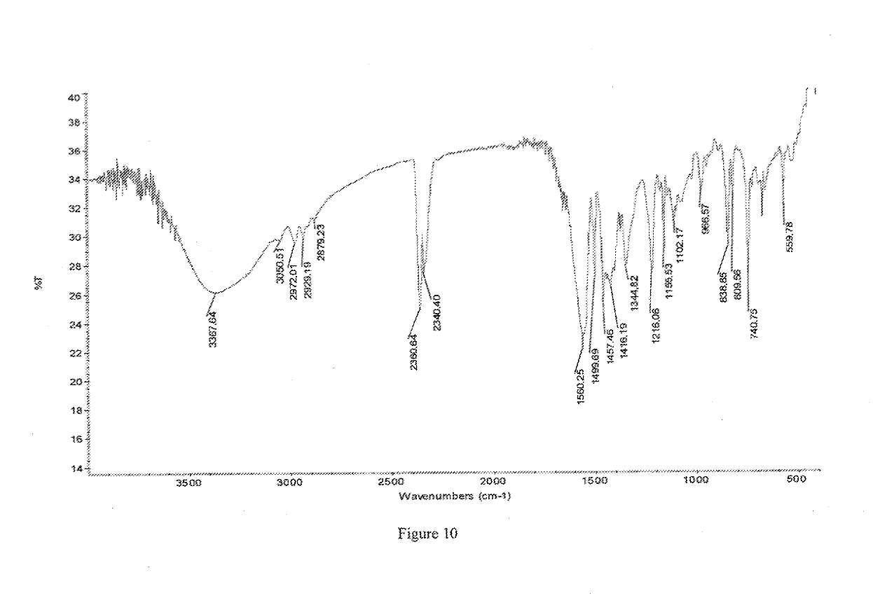

The fluvastatin calcium salt exhibits an IR spectrum (Sec FIG. 10) comprising peaks, for example, at about 2361, 1560, 1500, 1457, 1345, 1216, 1155, 839, 741, and about 560 cm⁻¹.

### Example 3

### Pharmacokinetic Study of Pravastatin Calcium Salt in Dogs

A two-way cross-over experiment was completed with six fasted beagle dogs to compare the pharmacokinetic parameters of pravastatin calcium salt with pravastatin sodium salt. The pravastatin sodium salt was acquired from PRAVACHOL® tablets. The pravastatin calcium salt was acquired via the method described in Example 1. The pravastatin calcium salt dosage form administered to the dogs consisted of 11.0 mg pravastatin calcium salt (equivalent to 10 mg pravastatin acid) and 744 mg Ropufa 75 ethyl esters of omega-3 fatty acids in a soft gelatin capsule shell. In vitro release testing of the capsules was completed and showed complete dissolution in deionized water at 37 degrees C. The mean dose of pravastatin free acid administered as PRAVACHOL® was 0.85 mg/kg and the mean dose of pravastatin free acid administered as pravastatin calcium salt was 0.95 mg/kg. Following administration, plasma samples were collected pre-dose and then at 0.25, 0.5, 1, 1.5, 2, 3, 4, 6, 8, 12, and 24 hours post-dose. Plasma samples were analyzed for pravastatin concentration using an LC/MS method. Table 2 shows several important pharmacokinetic parameters of pravastatin from both oral formulations dosed to six fasted beagle dogs.

TABLE 2
Pharmacokinetic parameters of pravastatin from two oral formulations
dosed to six fasted beagle dogs in a two-way cross-over study.

| | | | | | | |
|---|---|---|---|---|---|---|
| | AUC₀₋₁ | AUC_{inf} | | | | Relative |
| | (ng/mL × | ng/mL × | Cₘₐₓ | Tₘₐₓ | t_{1/2} | Bioavail- |
| Animal | hr) | (hr) | (ng/mL) | (hr) | (hr) | ability^{a} |

Pravastatin Calcium Salt

| | | | | | | |
|---|---|---|---|---|---|---|
| 1001 | 512.94 | 518.11 | 99.0 | 1 | 3.44 | 134 |
| 1002 | 264.11 | 268.01 | 87.2 | 0.5 | 1.85 | 62.1 |
| 1003 | 488.36 | 494.27 | 182 | 1 | 1.67 | 74.9 |
| 2001 | 438.55 | 453.69 | 131 | 0.5 | 2.5 | 99.7 |
| 2002 | 505.30 | 515.14 | 166 | 0.25 | 1.89 | 148 |
| 2003 | 380.68 | 396.20 | 182 | 1 | 2.76 | 200 |
| Mean | 431.66 | 440.90 | 141.20 | 0.71 | 2.35 | 121 |
| SD | 95.879 | 96.306 | 41.822 | 0.332 | 0.679 | 54.0 |
| % CV | 22.2 | 21.8 | 29.6 | 46.9 | 28.9 | 44.6 |

PRAVACHOL ®

| | | | | | | |
|---|---|---|---|---|---|---|
| 1001 | 333.39 | 345.11 | 94.0 | 0.5 | 2.44 | N/A |
| 1002 | 375.46 | 378.47 | 117 | 1 | 1.69 | N/A |
| 1003 | 575.07 | 581.85 | 192 | 1 | 1.79 | N/A |
| 2001 | 350.84 | 414.58 | 40.2 | 1 | 10.18 | N/A |
| 2002 | 297.63 | 312.21 | 129 | 1 | 2.59 | N/A |
| 2003 | 165.66 | 171.78 | 33.6 | 0.5 | 2.06 | N/A |
| Mean | 349.68 | 367.33 | 100.97 | 0.83 | 3.46 | — |
| SD | 132.89 | 134.27 | 59.345 | 0.26 | 3.31 | — |
| % CV | 38.0 | 36.6 | 58.8 | 31.0 | 95.8 | — |

^{a}Bioavailability calculated relative to PRAVACHOL ® AUC_{inf}, values were normalized for the doses received by each animal

In general, AUC and Cₘₐₓ values were slightly higher for pravastatin calcium salt compared with the values from the PRAVACHOL® tablet. Tₘₐₓ values are comparable between the formulations. As a result, the relative bioavailability of pravastatin following administration of pravastatin calcium salt (normalized for the doses administered) appears to be slightly higher than that of PRAVACHOL®. These results suggest that the suspension of pravastatin calcium salt in pharmaceutical omega-3 ethyl esters does not significantly influence the pharmacokinetic behavior of pravastatin.

### Example 4

### Pravastatin Magnesium Salt

To 3 mL of a 30.5 mass percent pravastatin sodium solution was added 0.7 mL of a 49.5 mass percent magnesium chloride solution. The solvent for both solutions was deionized water. Phase separation of the two liquids was observed within 30 minutes. Crystallization from the dense phase occurred overnight. Two solid phases (crystal habits) were collected: (A) a “fluffy” suspended phase at the top of the reaction vessel and (B) a dense solid phase at the bottom of the reaction vessel. The resultant salt was a 2:1 pravastatin to magnesium salt.

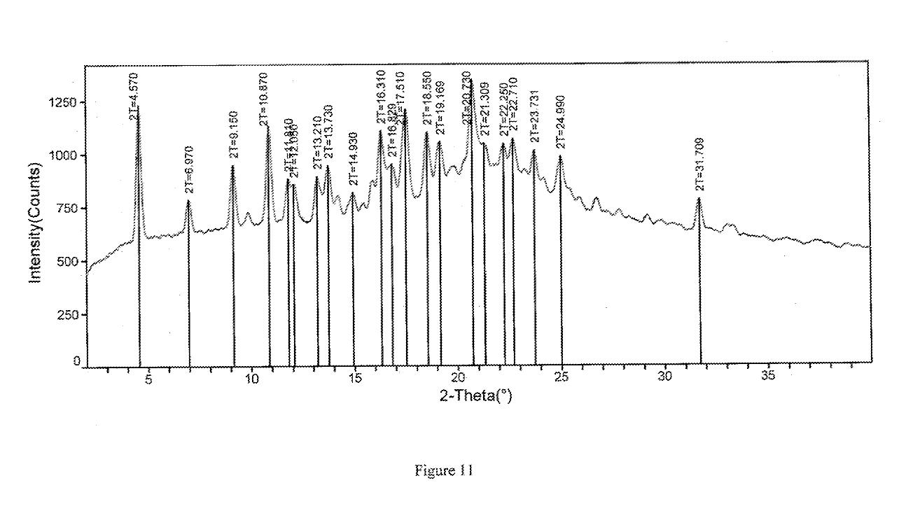

Crystals representative of those obtained by completing the method above were characterized using PXRD, DSC, TGA, IR, and DVS. The pravastatin magnesium salt (habit A) exhibits a PXRD diffractogram (See FIG. 11) comprising peaks, for example, at about 4.57, 6.97, 9.15, 10.87, 11.81, 13.21, 13.73, 16.31, 17.51, 18.55, 19.17, 20.73, 22.71, 23.73, and about 24.99 degrees 2-theta (Rigaku, data as collected). The peak observed at 31.709 degrees 2-theta corresponds to sodium chloride impurity.

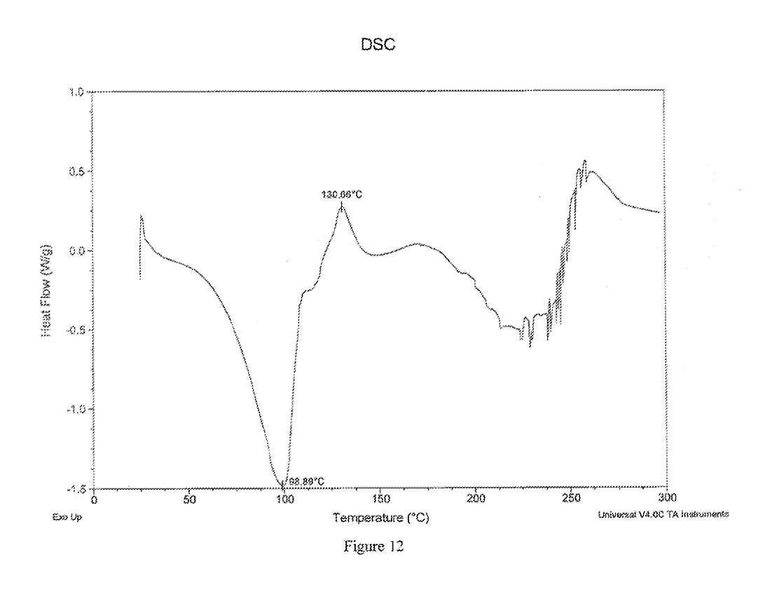

DSC was run (on pravastatin magnesium salt habit A) from 25 degrees C. to 300 degrees C. at 10 degrees C./minute. DSC showed an endothermic transition at about 99 degrees C. (See FIG. 12). The exotherm at about 131 degrees C. may represent a recrystallization event.

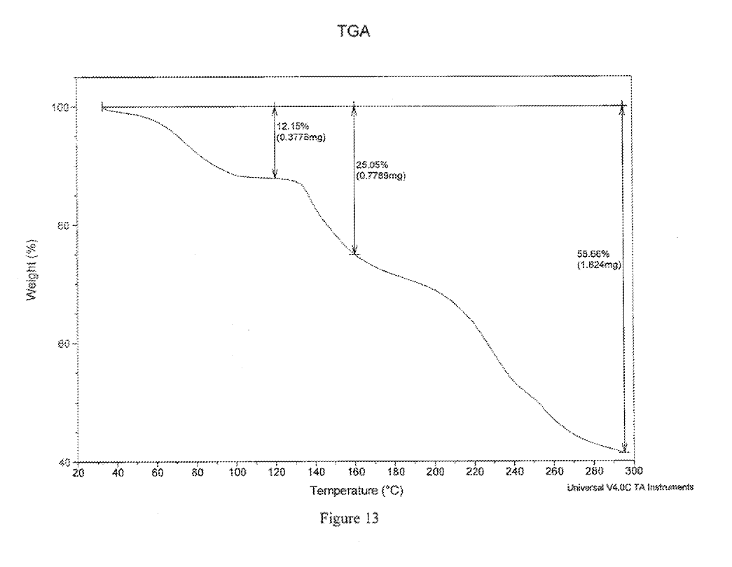

TGA was run (on pravastatin magnesium salt habit A) from 25 degrees C. to 300 degrees C. at 10 degrees C./minute. TGA showed about a 12 percent weight loss between 25 degrees C. and about 120 degrees C., and about a 25 percent weight loss between 25 degrees C. and about 160 degrees C. (See FIG. 13).

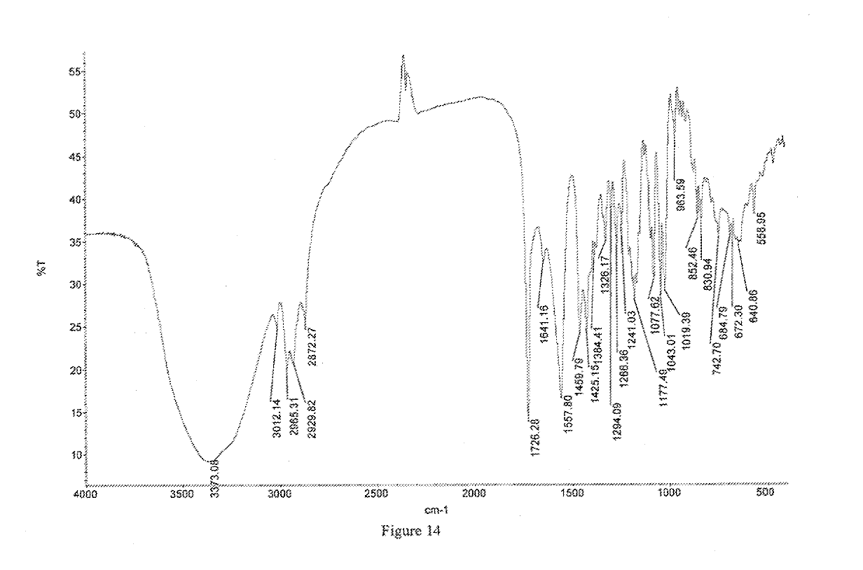

The pravastatin magnesium salt (habit A) exhibits an IR spectrum (See FIG. 14) comprising peaks, for example, at about 1726, 1557, 1425, 1177, 1078, 1019, and 641 cm⁻¹. The IR spectrum was acquired in transmission mode with the sample pressed into a KBr pellet. The spectrum is baseline corrected.

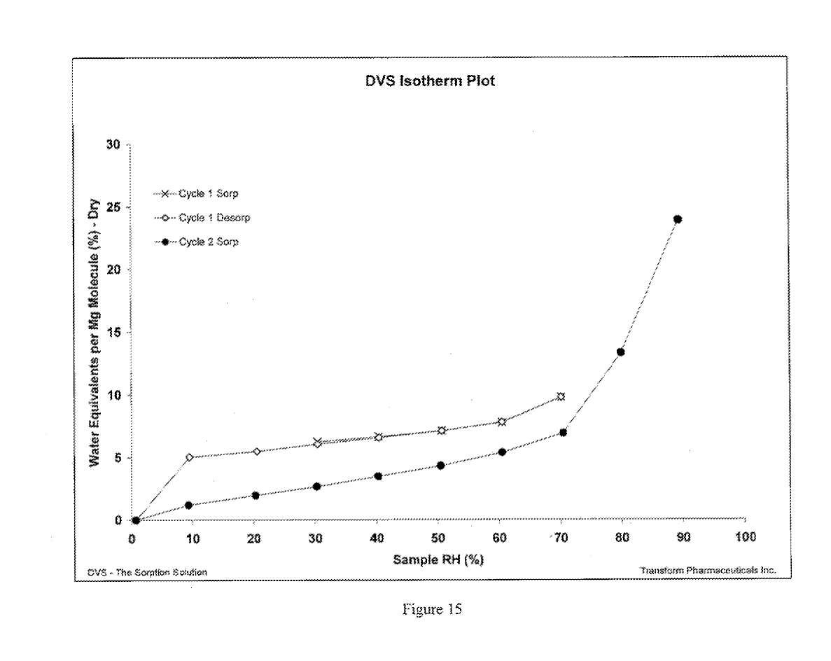

FIG. 15 shows a dynamic vapor sorption (DVS) isotherm plot of the pravastatin magnesium salt (habit A). This was completed at 25 degrees C. and the data show a stable region between about 10 and about 60 percent relative humidity (RH).

The solubility of pravastatin magnesium salt (habit A) in water was measured (via UV detection) to be 14.22 mg/mL.

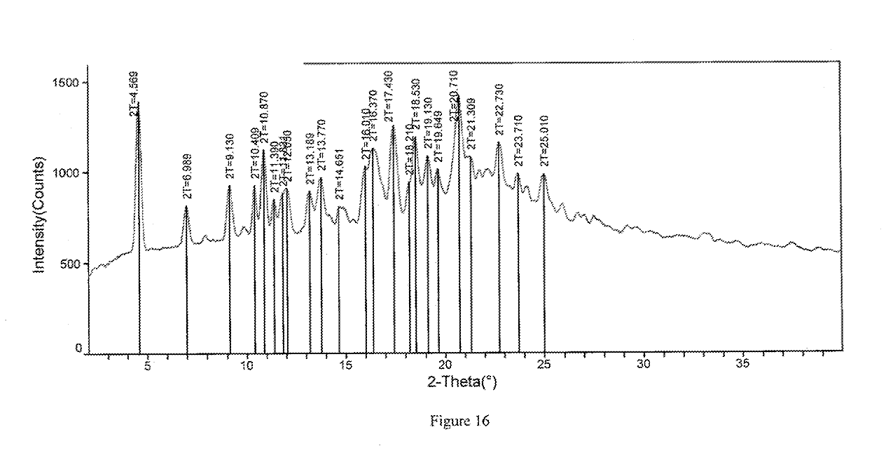

The pravastatin magnesium salt (habit B) exhibits a PXRD diffractogram (See FIG. 16) comprising peaks, for example, at about 4.57, 6.99, 9.13, 10.41, 10.87, 12.05, 13.19, 13.77, 16.37, 17.43, 18.53, 19.13, 20.71, 22.73, and about 25.01 degrees 2-theta (Rigaku, data as collected).

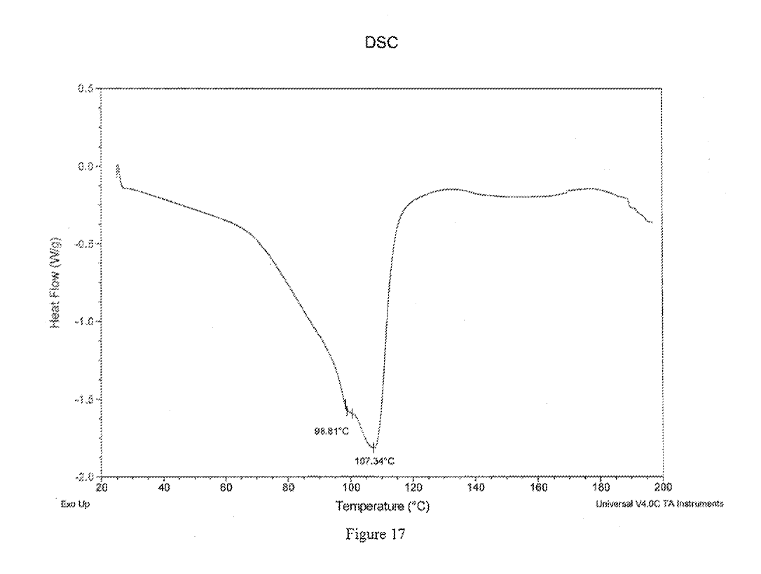

DSC was run (on pravastain magnesium salt habit B) from 25 degrees C. to 200 degrees C. at 10 degrees C./minute. DSC showed an endothermic transition at about 107 degrees C. (Sec FIG. 17).

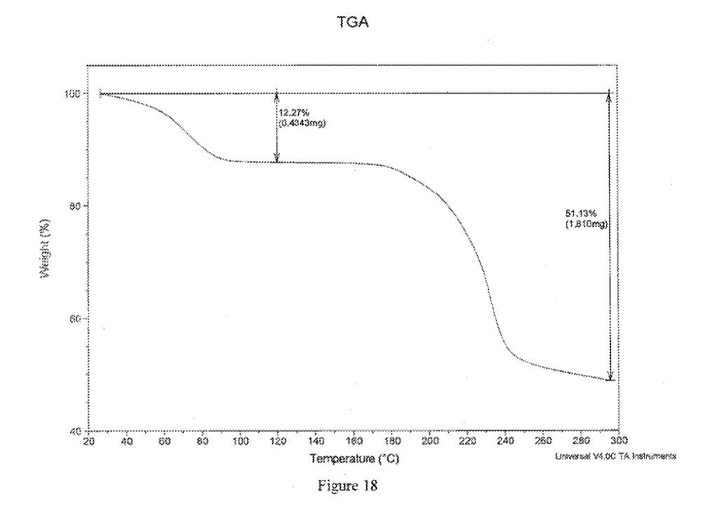

TGA was run (on pravastatin magnesium salt habit B) from 25 degrees C. to 300 degrees C. at 10 degrees C./minute. TGA showed about a 12 percent weight loss between 25 degrees C. and about 120 degrees C. (See FIG. 18).

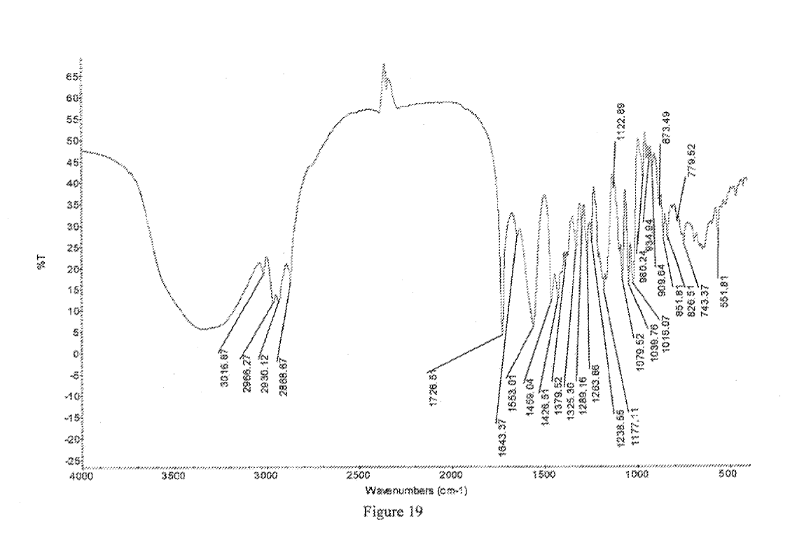

The pravastatin magnesium salt (habit B) exhibits an IR spectrum (See FIG. 19) comprising peaks, for example, at about 1726, 1553, 1459, 1426, 1177, 1079, 1039, and about 827 cm⁻¹. The IR spectrum was acquired in transmission mode with the sample pressed into a KBr pellet. The spectrum is baseline corrected.

The solubility of pravastatin magnesium salt (habit B) in water was measured (via UV detection, 20-25 degrees C.) to be 16.12 mg/mL.

### Example 5

### Pravastatin Magnesium Salt

Another preparation of pravastatin was completed: To a 49 mass percent solution of pravastatin sodium salt (1.0057 g; 2.25 mmol) in deionized water was added 2 molar equivalents of propylene glycol (0.171 g). Upon addition of a 53.1 mass percent magnesium chloride (230.0 g; 1.14 mmol) solution in deionized water, crystallization of pravastatin magnesium salt was noted. Overnight, the crystallization was observed to reach completion. The resultant salt was a 2:1 pravastatin to magnesium salt.

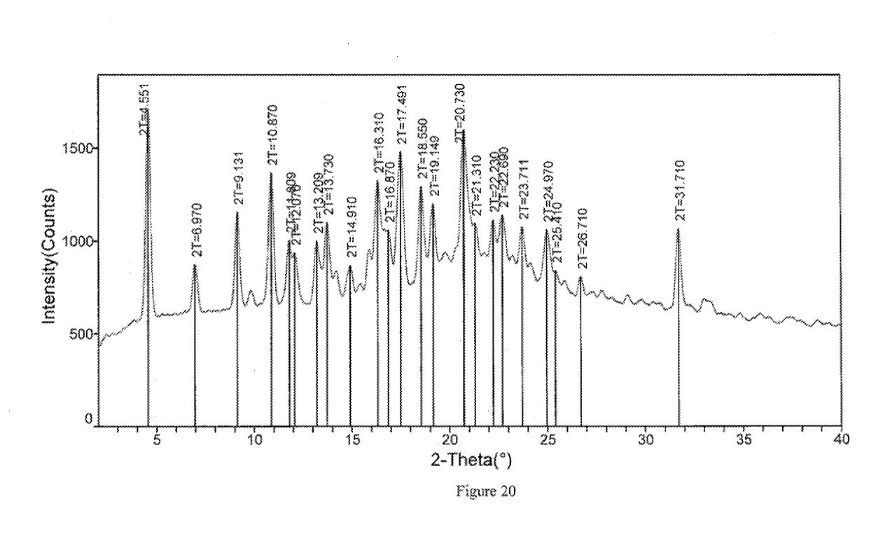

Crystals representative of those obtained by completing the method above were characterized using PXRD, DSC, and TGA. The pravastatin magnesium salt exhibits a PXRD diffractogram (See FIG. 20) comprising peaks, for example, at about 4.55, 6.97, 9.13, 10.87, 11.81, 13.21, 13.73, 16.31, 17.49, 18.55, 19.15, 20.73, 22.69, 23.71, and about 24.97 degrees 2-theta (Rigaku, data as collected). The peak observed at 31.710 degrees 2-theta corresponds to sodium chloride impurity.

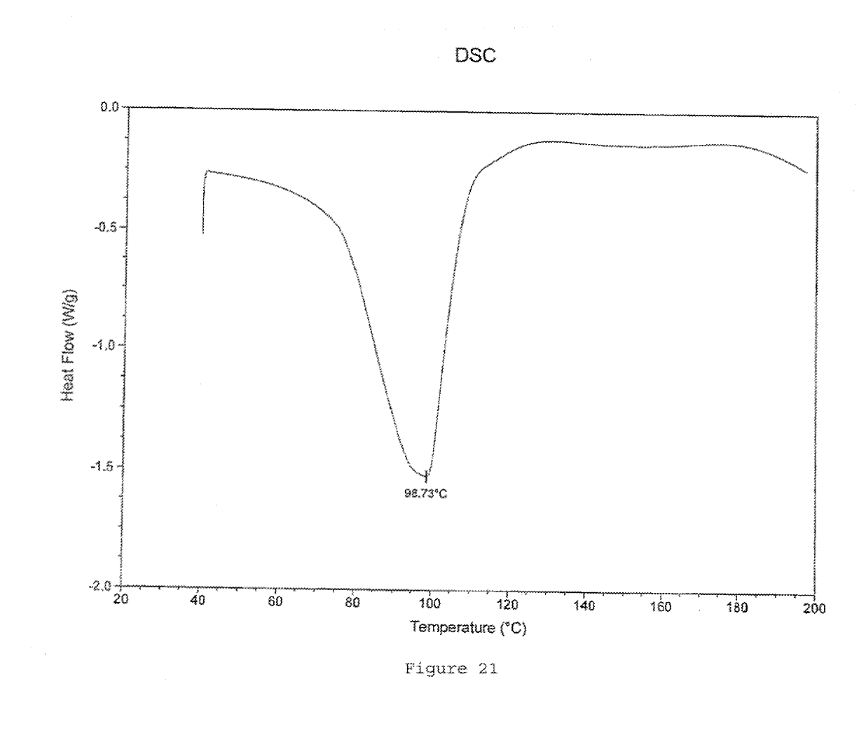

DSC was run (on pravastain magnesium salt) from 40 degrees C. to 200 degrees C. at 10 degrees C./minute. DSC showed an endothermic transition at about 99 degrees C. (See FIG. 21).

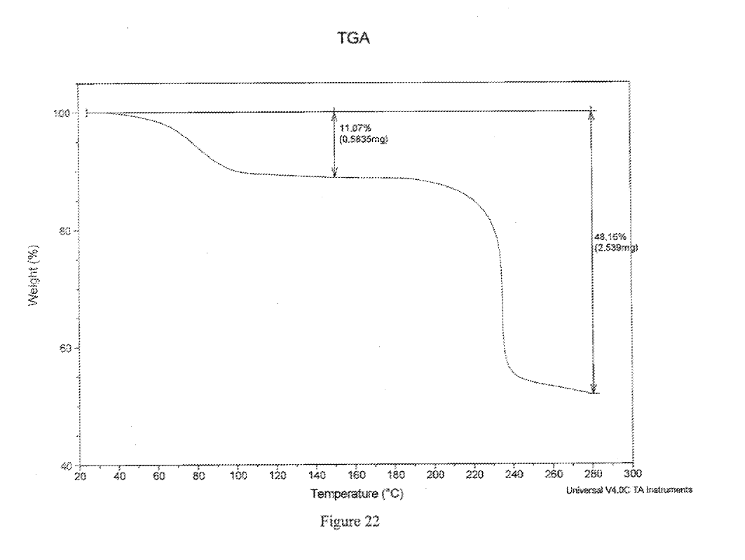

TGA was run (on pravastatin magnesium salt) from 25 degrees C. to 280 degrees C. at 10 degrees C./minute. TGA showed about an 11 percent weight loss between 25 degrees C. and about 150 degrees C. (See FIG. 22).

The solubility of pravastatin magnesium salt in water was measured (via UV detection, 20-25 degrees C.) to be 17.24 mg/mL.

### Example 6

### Pravastatin Zinc Salt

2 equivalents of pravastatin sodium dissolved in de-ionized water are reacted with a solution having 1 equivalent of zinc chloride in de-ionized water. Precipitation of crystalline pravastatin zinc occurs immediately at room temperature. The resultant salt was a 2:1 pravastatin to zinc salt.

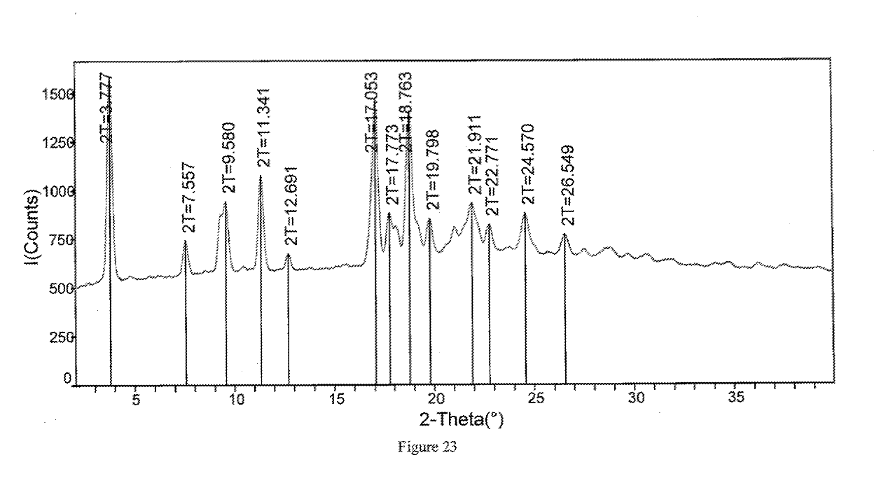

Crystals representative of those obtained by completing the method above were characterized using PXRD, DSC, TGA, IR spectroscopy, Raman spectroscopy, and DVS. The pravastatin zinc salt exhibits a PXRD diffractogram (See FIG. 23) comprising peaks, for example, at about 3.78, 7.56, 9.58, 11.34, 17.05, 18.76, 19.80, 21.91, 24.57, and about 26.55 degrees 2-theta (Rigaku, data as collected).

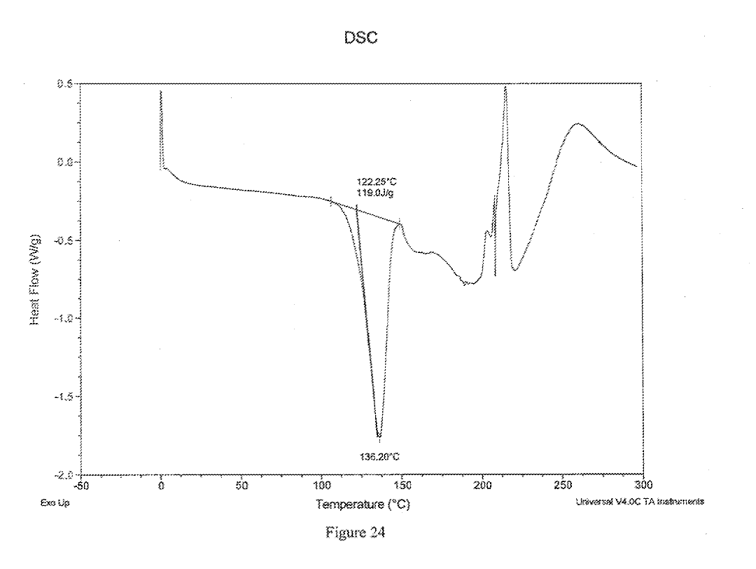

DSC was run (on pravastain zinc salt) from 25 degrees C. to 300 degrees C. at 10 degrees C./minute. DSC showed an endothermic transition at about 136 degrees C. (See FIG. 24).

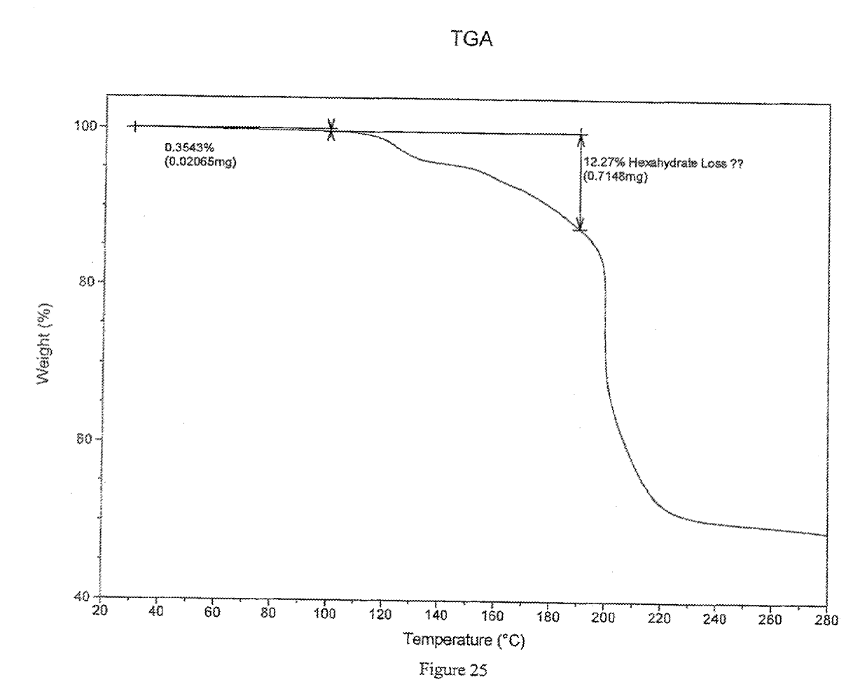

TGA was run (on pravastatin zinc salt) from 25 degrees C. to 300 degrees C. at 10 degrees C./minute. TGA showed about a 12 percent weight loss between about 100 degrees C. and about 190 degrees C., with negligible weight loss up to about 100 degrees C. (See FIG. 25).

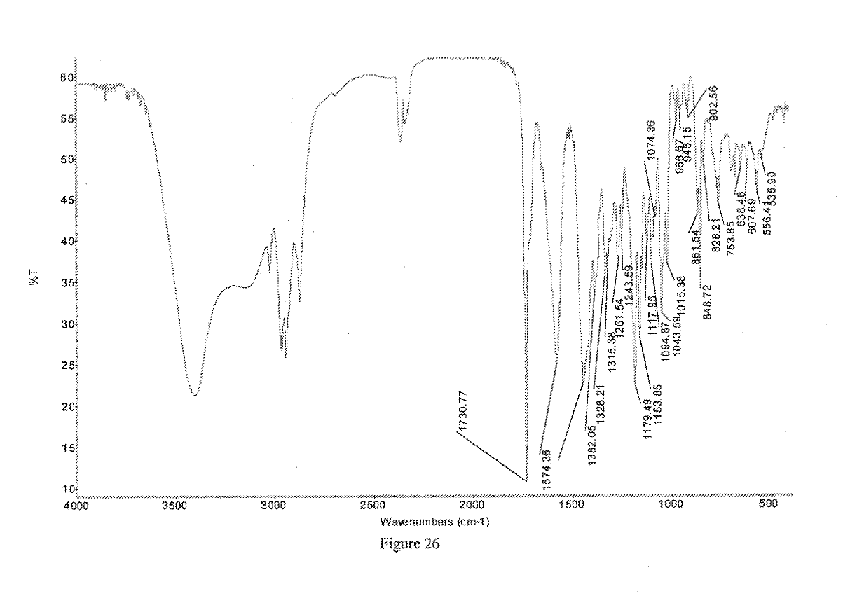

The pravastatin zinc salt exhibits an IR spectrum (See FIG. 26) comprising peaks, for example, at about 1731, 1574, 1179, 1044, 849, and about 754 cm⁻¹. The IR spectrum was acquired in transmission mode with the sample pressed into a KBr pellet. The spectrum is baseline corrected.

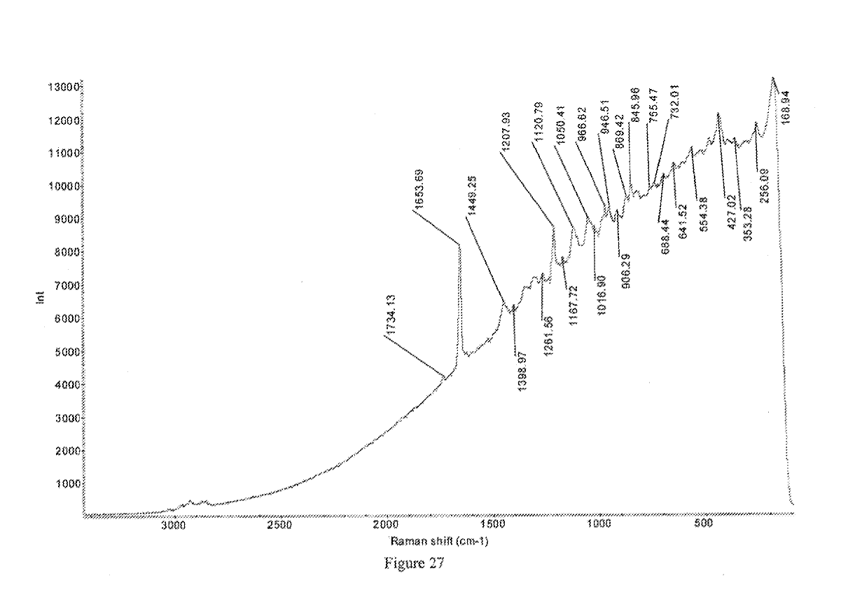

The pravastatin zinc salt exhibits a Raman spectrum (See FIG. 27) comprising peaks, for example, at about 1654, 1449, 1208, 1121, 1050, 846, and about 427 cm⁻¹.

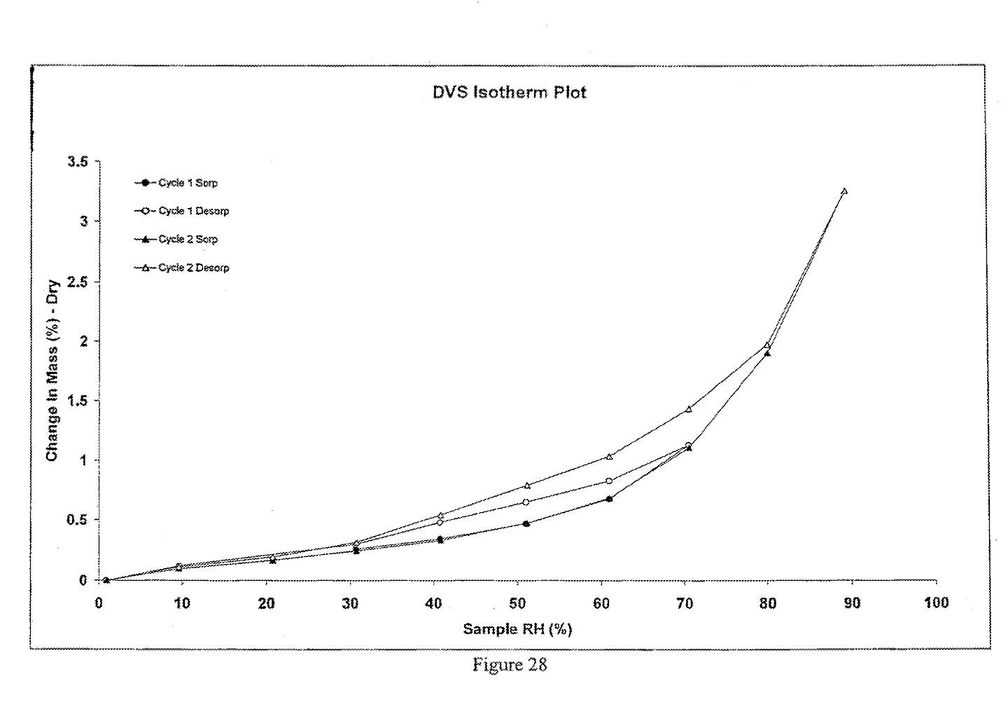

FIG. 28 shows a dynamic vapor sorption (DVS) isotherm plot of the pravastatin zinc salt. This was completed at 25 degrees C. and the data show a gradual increase in moisture sorption.

The solubility of pravastatin zinc salt in water was measured (via UV detection, 20-25 degrees C.) to be 0.53 mg/mL.

### Example 7

### 12 Week Stability Data of Pravastatin Salts in E681010:Ethanol Mixture

Several salts of pravastatin were suspended in 87:13 E681010:ethanol mixtures and placed in capped glass vials. Each suspension of pravastatin calcium, pravastatin magnesium, pravastatin sodium, or pravastatin zinc in 87:13 E681010:ethanol was measured periodically for 12 weeks. HPLC was used to measure degradation of the pravastatin salts.

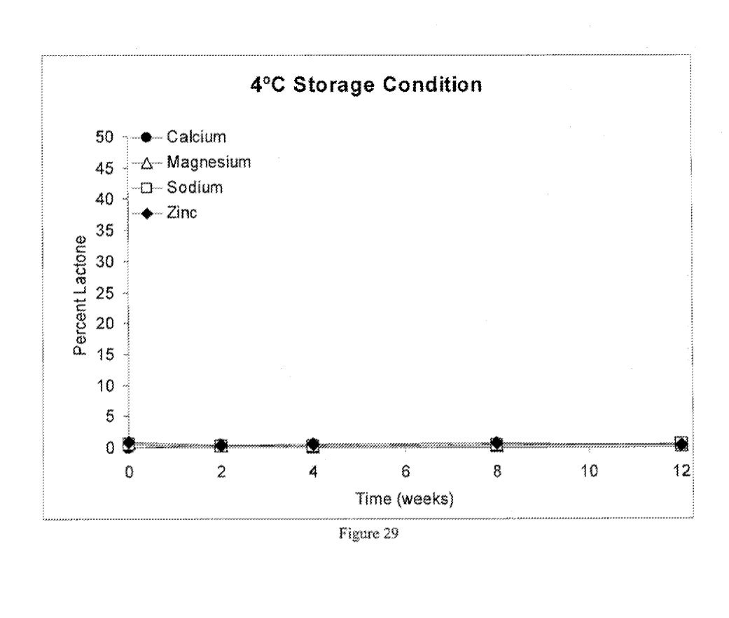

FIG. 29 shows the stability data (percent lactone) at 4 degrees C.

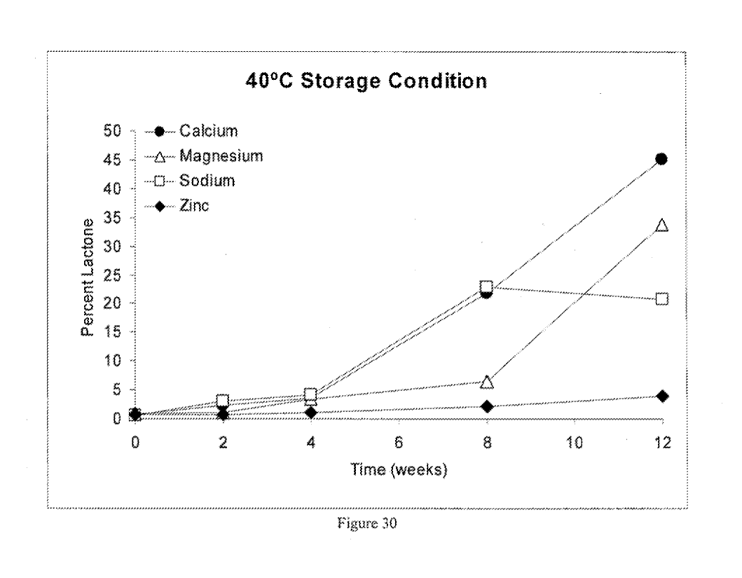

FIG. 30 shows the stability data (percent lactone) at 40 degrees C. The zinc salt exhibited the least degradation to the lactone with about 3 percent after 12 weeks.

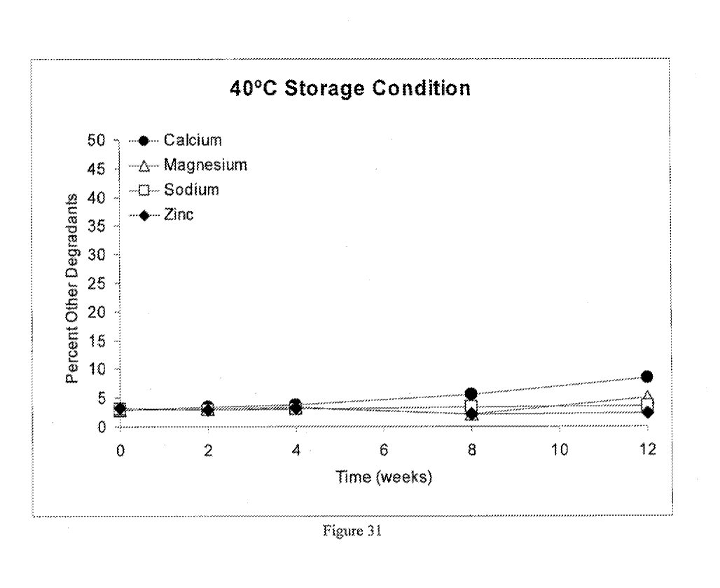

FIG. 31 shows the stability data (percent other degradants) at 40 degrees C. Again, the zinc salt appeared to be the most stable.

What is claimed is:

1. A pharmaceutical composition comprising:
an omega 3 oil;
a statin suspended in said omega 3 oil; and
a capsule;
wherein said statin has an aqueous solubility ranging between about 0.10 mg/ml and 25 mg/ml.

2. The pharmaceutical composition of claim 1, wherein said statin is a salt.

3. The pharmaceutical composition of claim 2, wherein said statin is a divalent salt.

4. The pharmaceutical composition of claim 3, wherein said statin is selected from the group consisting of fluvastatin calcium, atorvastatin calcium, rosuvastatin calcium, simvastatin calcium, cerivastatin calcium, and pravastatin calcium.

5. The pharmaceutical composition of claim 1, further comprising a surfactant.

6. The pharmaceutical composition of claim 1, wherein said omega 3 oil comprises at least 84% by weight of said composition.

7. The pharmaceutical composition of claim 1, wherein said omega 3 oil has a purity greater than about 85%.

8. The pharmaceutical composition of claim 1, wherein said composition contains about 1000 mg of omega 3 oil.

9. A formulation comprising:
about 1000 mg omega 3 oil;
a divalent salt of a statin; and
a capsule;
wherein said omega 3 oil has a purity greater than about 85% and wherein said formulation can be stored for 26 weeks at 25 degrees C. with no detectable degradation of said statin.

10. A pharmaceutical composition consisting essentially of:
an omega 3 oil;
a statin suspended in said omega 3 oil; and
a capsule;
wherein said statin has an aqueous solubility ranging between about 0.10 mg/ml and 25 mg/ml.
